(19) United States
(12) Patent Application Publication
(10) Pub. No.: US 2022/0249567 A1
(43) Pub. Date: Aug. 11, 2022

(54) LOW DENSITY CELL CULTURE

(71) Applicant: GLYCOSTEM THERAPEUTICS B.V., AB Oss (NL)

(72) Inventors: Volker HUPPERT, AB Oss (NL); Monica RAIMO, AB Oss (NL); Jan SPANHOLTZ, AB Oss (NL)

(73) Assignee: GLYCOSTEM THERAPEUTICS B.V., AB Oss (NL)

(21) Appl. No.: 17/629,257

(22) PCT Filed: Jul. 22, 2020
(86) PCT No.: PCT/NL2020/050478

§ 371 Date: Jan. 21, 2022

(30) Foreign Application Priority Data
Jul. 22, 2019 (NL) 2023550

(51) Int. Cl. A61K 35/17; C12N 5/0783; A61K 38/17; A61P 35/00

(52) U.S. Cl. CPC A61K 35/17; C12N 5/0646; A61K 38/1774; A61P 35/00; C12N 2506/025; C12N 2501/125; C12N 2501/145; C12N 2501/2306; C12N 2501/2307; C12N 2501/2315; C12N 2501/22; C12N 2501/26; C12N 2501/2302

(57) ABSTRACT

A method of producing a collection of natural killer cells from CD34⁺ human stem cells. The invention further provides to a collection of natural killer cells thus produced and a pharmaceutical composition having such, natural killer cells. Further, the invention relates to a method of using the pharmaceutical composition as a medicament, in particular for immunotherapy in the treatment of malignancies.

The invention relates to a method of producing a collection of natural killer cells from CD34⁺ human stem cells. The invention further relates to a collection of natural killer cells thus produced and a pharmaceutical composition comprising such natural killer cells. Further, the invention relates to the use of the pharmaceutical composition as a medicament, in particular for use in a method for immunotherapy in the treatment of malignancies.

### BACKGROUND

Haematopoiesis

Haematopoietic stem cells

The development of multicellular organisms is mainly dependent on the function of somatic stem cells. These cells are defined as undifferentiated cells, which can self-renew over a long period and give rise to progenitor cells committed to more specific lineages during development. Controlled development and differentiation of stem cells leads to a highly complex functional organ or organ systems. However, uncontrolled differentiation or genetic aberrations in stem cells could lead to death or development of cancer, immunodeficiency, autoimmunity or bone marrow insufficiency. In the last 50 years, HSC have become the most intensively studied stem cells regarding their self-renewal capabilities or lineage-specific differentiation and cell fate commitment on genetic and functional level. Furthermore, transplantation of HSCs has been extensively used to treat leukaemia and other types of cancers (1, 2). It has been clearly demonstrated that the adult and neonatal HSCs keep the ability to reconstitute the haematopoietic systems of patients after myeloablative treatment (3). Therefore, an important feature of HSCs is the capacity to replenish all lineages of mature blood cells.

From HSC to mature blood cells

Haematopoiesis describes the process and capacity of self-renewal of HSCs and the lifelong replacement of all distinct blood lineages representing progenitor and effector cells. Haematopoiesis develops during embryonic development and HSCs arise in mid-gestation within the region of the embryo that contains the dorsal aorta, gonads and mesonephros (i.e. the AGM region) and further develops in the yolk sac, placenta and foetal liver (4). Adult haematopoiesis is located in the bone marrow (BM) and the self-renewal and differentiation of HSCs is regulated within specific niches in the BM (5, 6). In the last 30 years, the most common model of haematopoietic development describes a binary cell fate tree of haematopoiesis, where in the first branch the HSCs gives rise to two different haematopoietic progenitor cells (HPCs), i.e. the common myeloid progenitor (CMP) for myeloid-erythroid cells (giving rise to erythrocytes, platelets, monocytes, macrophages and granulocytes) and the common lymphoid progenitor (CLP) for lymphoid cells (differentiating into T-, B-, and NK cells) (7).

Haematopoietic stem cell transplantation

Since more than 50 years HSCs are used for transplantation to treat haematological cancers and some solid tumours, following first line treatment with chemo- and radiotherapy in order to reduce tumour burden and achieve long term remission (8). As drug resistance and relapse remain major problems, autologous and human leukocyte antigen (HLA)-matched allogeneic HSCT are used as potentially curative cell therapy treatment for malignant and non-malignant haematological diseases. In allogeneic HSCT, donor T cells mediate a powerful graft-versus-tumour (GVT) effect (9). However, T cells can also cause GVHD and therefore limit the overall effectiveness of allogeneic HSCT. Various methods of T cell depletion reduce the risk of GVHD and allow in addition transplantation across the histocompatibility barrier but might increase the risk of graft rejection or relapse. Most interestingly, NK cells have been described to eliminate leukaemia relapse and graft rejection and to protect patients against GVHD in a haploidentical HSCT setting (10), mainly by their ability to inhibit and lyse GVHD inducing T cells (11) and host antigen presenting cells (APCs), which are critical for the activation of donor T cells in GVHD induction (12). Furthermore, there is clinical evidence, that high NK cell doses in unrelated HSCT prevent severe GVHD, while preserving the GvT effect (13). Therefore, NK cells have become nowadays a very attractive lymphocyte population for anticancer immunotherapy in HSCT and non-HSCT transplant related treatment schemes.

Natural Killer cells NK cell subsets and function NK cells are originally identified in the mid 70′s as the third major subpopulation of lymphocytes, beside T- and B-cells (14-17). NK cells are important effector cells of the innate immune system because they can exert rapid effector function without prior sensitization, i.e., “natural” killing. Therefore, NK cells play a key role in early defence against viral and bacterial infections and in tumour immune surveillance (18, 19). NK cells are present in lymphoid organs and various non-lymphoid tissues. Beside their cytolytic activity, NK cells are able to produce a wide variety of cytokines and chemokines to influence the other cellular compartments of the immune system (20, 21). NK cells can be broadly defined as CD56⁺CD3⁻ lymphocytes comprising 5-15% of the circulating lymphocyte population (22). They can be subdivided into two major subsets based on their CD56 expression levels. CD56^{dim}NK cells, accounting for approximately 90% of peripheral blood NK cells have marked cytolytic potential and express high levels of the low affinity Fc receptor III (FcRγIII; recognized by CD16) allowing them to mediate antibody-dependent cellular cytotoxicity (ADCC) (23, 24). In contrast, CD56^{bright} NK cells, representing ˜10% of all NK cells, have predominantly immune regulatory functions mediated by a potent production of INF-γTNF-αand GM-CSF.

NK cells recognize and kill infected or malignant-transformed cells through signals from germ line-encoded inhibitory receptors (IR) or activating receptors (AR) (25). The combination of these signals balances and modulates NK cell effector functions. When the activating signals dominate, NK cell cytotoxicity and cytokine production are triggered. In contrast, these functions are blocked when inhibition is predominant. Upon activation, NK cells lyse susceptible target cells using several killing mechanisms (26), including release of cytotoxic granules containing perforin and granzymes (27, 28), TRAIL-dependent cytotoxicity and activation of Fas-mediated apoptosis (28, 29).

NK cell receptors

NK cells use a variety of ARs and IRs to recognize tumour or virus-infected cells (30-32). In humans, several types of HLA class I-specific IRs have been described including the killer inhibitory immunoglobulin receptors (KIRs) that recognize groups of HLA class I molecules, and c-type lectin receptors of the CD94/NKG2A family specific for the non-classical HLA-E molecule presenting peptides derived from signal sequences of classical HLA class I molecules (33, 34). The discovery of these receptors emerged from early observations showing that NK cell cytotoxicity was triggered by tumour cells lacking self MHC class I molecules, which is referred as “missing-self” recognition. Each NK cell expresses a different combination of inhibitory and stimulatory receptors such that at least one inhibitory KIR specific for a self MHC class I allele is present. In steady state, NK cells are inhibited by the recognition of self-HLA class I molecules that overrule potential stimulatory signals. But in case of malignant transformation, tumour cells may down regulate self HLA class I expression or display only non-self HLA class I molecules in the HLA-mismatched transplantation setting, while up-regulating activating ligands triggering NK cell-mediated tumour cell lysis. In a HSCT setting, where donor NK cells miss an inhibitory KIR for an HLA ligand present on a recipient tumour cell, NK cells are thought to exert higher GVT effect, because they are potentially less inhibited by the existing “KIR-ligand” mismatch. Retrospective studies in the setting of haploidentical allogeneic HSCT, cord-blood (35) and allogeneic adoptive NK cell therapy have shown that this “KIR-ligand” mismatch could be responsible for the anti-tumour effect.

The activating signals are mediated by ARs of which the most important receptors, beside CD16 described above, are NKG2D, DNAM-1 and the natural cytotoxicity receptors (NCR); NKp30, NKp44 and NKp46. The ligands for NKG2D are the stress-inducible proteins MICA/B and ULBPs. DNAM-1 recognizes the poliovirus receptor (PVR) and nectin-2, whereas the ligands for the NCRs are heparin, heparin sulphates and viral hemagglutinin, but there are also potential proteins involved in the recognition by NCRs that have not been identified so far. Most of the ligands for these ARs are expressed predominantly by “stressed” cancerous and virus-infected cells. High expression of activating ligands in combination with high expression of ARs on NK cells can overcome the inhibitory signal and activate the NK cell to kill target cells. Based on these functional concepts, NK cells can induce tumour cell death without prior immunization as well as produce cytokines such as IFN-γTNF-α and GM-CSF that are key mediators in activating dendritic cells in lymph nodes thereby linking innate NK cell-based immunity to adaptive T cell-mediated immunity. Interestingly, upon stimulation with cytokines such as IL-2, IL-15, IL-18, IL-12 or IFN-γ, NK cell cytotoxic activity increases and these activated NK cells are able to eradicate the targets that are resistant to the so called “resting NK cells”(/8).

NK cell development

NK cells arise from HSCs and the BM is generally considered as the primary site for human NK cell development (4-7) NK cells and their progenitors are also present in lymphoid organs such as spleen, liver and lymph nodes, but can also migrate to the lung, gut and various other tissues (reviewed in (36)). Within the BM microenvironment, CD34⁺ HSCs and HPCs can be forced by growth factors like IL-2 or IL-15 to differentiate into the NK cell lineage. In 2005, Freud et al. identified a BM-derived CD34⁺ HPC residing in lymph nodes (LN) where further differentiation into CD56^{bright} NK cells could take place (37). Furthermore, they identified four discrete stages of human NK cell development within secondary lymphoid tissues (SLT) based on cell surface expression of CD34, CD117 and CD94: i.e. stage 1, CD34⁺CD117⁻CD94⁻; stage 2, CD34⁺CD117⁺CD94⁻; stage 3, CD34⁻CD117+CD94⁻; and stage 4, CD34⁻CD117^{+/-}CD94⁺(38). Following NK cell development, commitment to the NK cell lineage takes place at stage 3, in which CD56 appears on the cell surface and gives rise to CD56^{bright} NK cells in stage 4. These data confirmed previous studies describing the abundant presence of CD56^{bright} NK cells in SLT (39, 40). In addition, it has been shown that CD56^{bright} cells are the first mature NK cells to arise after haematopoietic SCT (41, 42). Overall, these data support a model of in vivo human NK cell development in which CD34⁺ NK cell precursor traffic from BM to SLT where further differentiation into CD56^{bright} NK cells takes place.

Up to date, a complete pathway for human NK cell development and maturation has not been described and it could be possible that NK cell precursors traffic from BM to other tissues for terminal differentiation in situ (43). Furthermore, NK cells acquire their cytotoxic and cytokine-production capability through a process called “NK cell licensing or education” (44, 45). Although this process remains to be completely understood, it is supposed that NK cells become functional competent (i.e. “licensed to kill”) after engagement of IRs with self-HLA class I ligands during the education process (44, 46). Based on this “licensing” model, NK cells without expression of IRs do not complete the education process, therefore remaining unlicensed and functionally “hyporesponsive” (46-48). During the described transition of NK cells through several stages of the NK cell developmental pathway, stage 3 cells transit into stage 4 cells by acquiring CD94/NKG2A receptors. These cells are then capable to react to certain cytokines such as IL-2, IL- 12, IL-15, IL-18 or IL-21 in order to acquire cytokine-producing functions and low cytotoxic potential (49, 50). Thereafter, fully educated NK cells acquire the ability to migrate to peripheral tissues and additionally obtain functional receptors like CD16, NKG2D or KIRs to become highly cytotoxic (51, 52).

Several NK cell progenitors have been identified in the pool of CD34⁺ HSCs and HPCs, and initially NK cells were thought to develop from a bipotent progenitor for T and NK cells (53). Most in vitro studies on NK cell development have been performed by culturing purified human CD34⁺ cells from BM(54), PB, CB (55) and foetal liver (FL) (56), mainly in the presence of IL-15, IL-2, SCF and Fms-related tyrosine kinase 3 ligand (F1t3-L), which could be differentiated into CD56⁺CD3⁻NK cells. This approach has identified additional surface antigens on NK cell progenitors including CD7 (57), CD122 (58), CD161 (59)and CD45RA^{high} (37). Furthermore, CD34⁺CD38⁻CD7⁺ cells were found to be the most primitive lymphoid precursor cells that give rise to NK cells, B cells and DCs, but not myeloid or erythroid cells. However, also myeloid-like CD14+CD11b+CD13+CD33+ cells (60) or CD56⁻CD117⁺M-CSFR⁺ cells (61) have been recently described with the capability to differentiate into mature and functional NK cells using IL-15 and Flt3-L or stromal feeder cells. Mainly studies based on mouse NK cells and mouse models have identified various transcription factors playing a role in regulating NK cell development such as Ets-1 (62), PU.1 (63), MEF (64), GATA-3 (65), T-bet (66), Irf-2 (67), Id2 (68, 69), E4BP4 (70), however most of these transcription factors do not play an exclusive role in NK cell development as shown in knockout experiments.

The majority of in vitro expansion and differentiation protocols thus make use of the CD34⁺ stem cells that are present in the sourcing material (e.g. BM, PB, CB or foetal liver). Upscaling of NK cell production is largely hampered by the low percentage of CD34⁺ stem cells in these tissues, and thus low absolute cell count of these cells to start with. For example, CD34⁺ stem cells can be isolated from peripheral blood, using an apheresis machine after being mobilized from the bone marrow to the blood, in a quantity of about 1,5×10⁵/ ml (71). In a single apheresis, up to 7×10⁸ CD34⁺ cells can be obtained (71, 72). Starting from cord blood, even lower total cell count is to be expected, in median about 1.5×10⁶ total from one donor, with high variability. Yield of CD34⁺ haemopoietic stem cells from cord blood has been shown to vary with gestational age (73), mode of delivery (74) and positioning of the delivered neonate after delivery (75). Due to the low cell count and high variability therein, only about 20% of all cord blood units, so called “high output” cord blood units, provide sufficient starting material for our previously described and validated NK cell expansion and differentiation protocol in order to obtain sufficient NK cells to be used in clinical trials (WO2017077096). As a result, only every fifth cord blood unit can be used for production of clinical batches.

Although cord blood yields the lowest absolute number of CD34⁺ cells as starting material for expansion and differentiation into NK cells, cord blood is preferred given its ease of collection, relative immune tolerance, unlimited supply, and lack of ethical concerns (76). However, as described above, low CD34⁺ cell numbers per collection pose a distinct disadvantage, especially for larger sized adult transplant recipients. Methods to enhance absolute CD34⁺ numbers without altering cell quality within each cord component are vital to their continued use in clinical studies. Such studies have been reviewed by others (76) and show an up-to 330-fold increase in CD34⁺ absolute numbers, e.g. using StemRegeninl after 15 days of culture (77). Nicotinamide, e.g., shows a 21-fold increase after 21 days (78) and mesenchymal stem cell co-culture a thirty-fold increase after 14 days (79). The addition of chemical compounds or feeder cells to the (initial) culture may, however, have implications on the further development and differentiation of the NK cells. It may also have implications for obtaining marketing authorization.

Therefore, new methods of increasing the number of CD34⁺ stem cells prior to differentiation and/or methods of increasing the number and/or quality of the ultimate differentiated NK cells are required.

### SUMMARY OF THE INVENTION

The present invention provides such method(s) for increasing the yield of CD34⁺ derived NK cells. In one aspect, the invention provides a method for producing a collection of stem cells, progenitor cells, and/or progenitor NK cells, said method comprising the step of

(i) initiating a cell culture from a sample comprising CD34⁺ human stem cells and culturing the cells for at least 7 days in a basic culture medium comprising stem cell factor (SCF) and interleukin −7 (IL-7), and one or more of flt-3Ligand (FLT-3L) and thrombopoietin (TPO), characterized in that the cell culture comprising CD34⁺ human stem cells is initiated at a cell density of 10,000 CD34⁺ cells/ml or less, preferably between 500 and 10,000 CD34⁺ cells/ml, more preferably between 1,000 and 8,000 CD34⁺ cells/ml, more preferably between 2,000 and 6,000 CD34⁺ cells/ml. Subsequently, the culturing step may be followed by a step of culturing cells obtained in step (ii) for at least 4 days in a culture medium comprising a collection of cytokines, wherein said collection of cytokines comprises three or more of IL-15, IL-2, SCF, and IL-7, thereby obtaining a collection of cultured cells containing a plurality of NK progenitor and/or NK cells.

A method according to the invention does not only result in an absolute increase of haematopoietic stem or progenitor cells after 12-15 days of culture, but it was surprisingly found that further expansion and differentiation culture methods (as described in the Examples and previously in WO2017077096) led to a further increase in expansion and to an equal or better quality of NK cells than a method wherein the CD34⁺ stem cells were cultured in a more dense concentration). One further advantage of a method according to the invention is that it enables culturing CD34⁺ haematopoietic stem cells obtained from automatic cell sorters without prior manual handling such as, e.g., centrifugation and resuspension in smaller volumes. This is because the cell concentration after automatic cell sorting is in the range of about 500-10,000 CD34⁺ cells/ml, whereas conventionally about 100,000 CD34⁺ cells/ml or more are initially cultured (Veluchamy et al, Front Immunol 2017, 8: 87; Roeven et al, Stem Cells and Development 2015, 24(24): 2886-2898). This enables further automation of the selection and culturing process, which is beneficial for standardization and obtaining marketing authorization.

In one aspect, therefore, the invention provides a method for producing a collection of stem cells, progenitor cells, and/or NK cells, said method comprising the step of
selecting CD34⁺ HSC through a fully automated closed system;
collecting and culturing the thus selected CD34⁺ HSC from a sample comprising CD34⁺ human stem cells and culturing the cells for at least 7 days in a basic culture medium comprising interleukin-7 (IL-7) and stem cell factor (SCF), and one or more of flt-3Ligand (FLT-3L) and thrombopoietin (TPO), wherein the collecting step is preferably within the fully automated closed system, characterized in that the cell culture is initiated at a cell density of at most 12,000 CD34⁺ cells/ml, preferably between 500 and 10,000 CD34⁺ cells/ml, more preferably between 1,000 and 8,000 CD34⁺ cells/ml, most preferably at a cell concentration of between 2,000 and 6,000 CD34⁺ cells/ml . The method may further be extended by performing a subsequent culturing step of culturing cells obtained in step (ii) for at least 13 days in a culture medium comprising a collection of cytokines, wherein said collection of cytokines comprises three or more of IL-15, IL-2, SCF, and IL-7, thereby obtaining a collection of cultured cells containing a plurality of NK progenitor and/or NK cells.

NK cells thus obtained are different from NK cells known to date in that they are, inter alia, in a very high percentage fully differentiated after 28 days or 35 days of culture, are able to efficiently kill target cells, and can be obtained in very high cell numbers from one single donor. Therefore, in one aspect, the invention also provides a collection of NK cells obtained by a method according to the invention, wherein the collection of NK cells has at least one or more of these novel and inventive properties. In one aspect, the collection of NK cells according to the invention is characterized in that it contains at least 10,000,000,000 (10¹⁰) NK cells from a single donor.

In one aspect, the invention provides a pharmaceutical composition comprising a (part of the) collection of the novel and inventive NK cells. The pharmaceutical composition is especially useful as a medicament, in particular for use in the treatment of tumours and haematological malignancies.

In one aspect the invention provides a method of treating an individual in need of adoptive NK cell transfer, the method comprising administering to said individual (part of) the collection of the novel and inventive NK cells.

### BRIEF DESCRIPTION OF THE FIGURES

FIG. 1—Influence of seeding density on the expansion of CD34⁺ haematopoietic stem cells (HSCs) purified from fresh umbilical cord blood (UCB) and cultured in complete expansion medium. A. Freshly isolated day 0 HSCs are cultured in expansion medium at different, increasing densities and cell expansion is analysed via flow cytometry on days 5-6, 8-9 and 12-15. Similar cell densities are grouped: 500 CD34⁺ cells/ml, 2000 CD34⁺ cells/ml and 3000 CD34⁺ cells/ml: <3000 CD34⁺ cells/ml; 10000 CD34⁺ cells/ml; 30000 CD34⁺ cells/ml and 40000 CD34⁺ cells/ml: 30-40000 CD34⁺ cells/ml; 60000 CD34⁺ cells/ml and 75000 CD34⁺ cells/ml: 60-75000 CD34⁺ cells/ml; 100000 CD34⁺ cells/ml and >250000 CD34⁺ cells/ml. The cell expansion, calculated as [cells/ml at day of measurement divided by CD34⁺ cells/ml at day 0] for every condition, is plotted against time. Low density-seeded cells, i.e. up to 10000 CD34⁺ cells/ml, show higher expansion potential. B. HSCs relative endpoint (day 12-15) expansion is calculated for each cell density on 100000 CD34⁺ cells/ml (set as =1) and plotted against the increasing density, where 500 CD34⁺ cells/ml is set as =1 (i.e., 1-6=<3000 CD34⁺ cells/ml, 20=10000 CD34⁺ cells/ml, 60-80=30-40000 CD34⁺ cells/ml, etc.). The histogram shows an inverse correlation between the two variables, indicating how the high expansion potential of low density seeded cells progressively reduces when cell density increases. Results are the mean (SD) of technical duplicates or triplicates for N=5 biological replicates; data are analysed using 1-way ANOVA. * p≤0.033; ** p≤0.002; *** p≤0.001; all other differences observed are statistically non-significant.

FIG. 2—Influence of expansion phase density on CD34⁺ haematopoietic stem cells (HSCs)-derived progenitors' differentiation into mature effector cells in complete differentiation medium. On day 12-15, cell culture setting is switched from expansion to differentiation phase; all conditions are set at the same density of 1.5*10⁶cells/ml and cell differentiation phenotype is monitored via flow cytometry at days 20-21, 27-28 and 35-36. Similar cell densities are grouped: 500 CD34⁺ cells/ml, 2000 CD34⁺ cells/ml and 3000 CD34⁺ cells/ml: <3000 CD34⁺ cells/ml; 10000 CD34⁺ cells/ml; 30000 CD34⁺ cells/ml and 40000 CD34⁺ cells/ml: 40000 CD34⁺ cells/ml; 60000 CD34⁺ cells/ml and 75000 CD34⁺ cells/ml: 75000 CD34⁺ cells/ml; 100000 CD34⁺ cells/ml. The plot shows the progression of the differentiation (as % of differentiated cells in culture) against time. Low density conditions (<3000 CD34⁺ cells/ml) are slightly delayed during early differentiation (day 20-21). At day 35-36, all conditions are fully differentiated into effector cells, with low densities (<3000 CD34⁺ cells/ml and in particular 10000 CD34⁺ cells/ml) reaching slightly higher and more consistent numbers. Results are the mean (SD) of technical duplicates or quadruplicates for N=5 biological replicates; data are analysed using 1-way ANOVA. Any differences observed are statistically non-significant.

FIG. 3—Influence of expansion phase density on mature effector cells potency in complete medium. The potency of effector cells, determined as the % of killed target tumour cells after overnight co-culture at a 1:1 effector:target ratio, is analysed via flow cytometry for all expansion densities at day 35-36. The plot shows the % of killed target cells for all densities, grouped as follows: 500 CD34⁺ cells/ml, 2000 CD34⁺ cells/ml and 3000 CD34⁺ cells/ml: <3000 CD34⁺ cells/ml; 10000 CD34⁺ cells/ml; 30000 CD34⁺ cells/ml and 40000 CD34⁺ cells/ml: 40000 CD34⁺ cells/ml; 60000 CD34⁺ cells/ml and 75000 CD34⁺ cells/ml: 75000 CD34⁺ cells/ml; 100000 CD34⁺ cells/ml. Cell potency is higher for low density conditions (10000 CD34⁺ cells/ml, and in particular <3000 CD34⁺ cells/ml) and progressively reduces with increased density. Results are the mean (SD) of technical triplicates for N=4 biological replicates; data are analysed using 1-way ANOVA. Any differences observed are statistically non-significant.

FIG. 4—Prodigy- and manually-selected CD34⁺ haematopoietic stem cells (HSCs) and effector cells morphology during cell culture. Visual appearance and proliferation of HSCs and effector cells is monitored during the expansion and the differentiation phases; the 75000 CD34⁺ cells/ml density is shown for both Prodigy and manual selection methods as representative of all expansion conditions. Cells stay healthy, rounded and do not cluster through expansion; intensive proliferation is appreciable from day 2 (panels a, b) through day 9 (panels c, d) until day 14 (panels e, f). Red blood cells present in culture at day 2 have completely disappeared at day 14. Differentiated effector cells at day 35 look healthy and pear-shaped (panels g, h) for both methods. Day 2, 9 and 14 pictures are taken from the same cell culture well for both conditions; day 35 pictures are taken from the same cells after differentiation. N=1 biological replicate.

FIG. 5—Influence of cell isolation method and seeding cell density on the expansion of CD34⁺ haematopoietic stem cells (HSCs) purified from fresh umbilical cord blood (UCB) and cultured in complete expansion medium. A. Freshly Prodigy- and manually-isolated day 0 CD34⁺ HSCs are cultured in expansion medium at 6 different, increasing densities (500 CD34⁺ cells/ml, 2000 CD34⁺ cells/ml, 10000 CD34⁺ cells/ml, 40000 CD34⁺ cells/ml, 75000 CD34⁺ cells/ml, 100000 CD34⁺ cells/ml) and cell expansion is analysed via flow cytometry at day 15. Cell expansion, calculated as [cells/ml at day 15 divided by CD34⁺ cells/ml at day 0] for every condition, is plotted for day 0 and day 15. Low density seeded cells show higher expansion potential, with no difference between the two methods. B. Prodigy- and manually selected cells relative day 15 expansion is calculated for each cell density on 100000 CD34⁺ cells/ml (set as =1) and plotted against the increasing density, where 500 CD34⁺ cells/ml is set as =1 (i.e., 1=500 CD34⁺ cells/ml, 4=2000 CD34⁺ cells/ml, 20=10,000 CD34⁺ cells/ml, etc.). The histogram shows an inverse correlation between density and expansion, with no difference between the two methods. Results are single data points or average of duplicates. N=1 biological replicate.

FIG. 6—Influence of cell isolation method and expansion phase density on CD34⁺ haematopoietic stem cells (HSCs)-derived progenitors' differentiation into mature effector cells in complete differentiation medium. On day 15, Prodigy- and manually-isolated cells, expanded at different densities, are seeded at 1.5*10⁶ cells/ml, and cell culture conditions switched from expansion phase to differentiation phase. Cell differentiation progression is monitored via flow cytometry at days 20, 28 and 35. The plots show the progression of the differentiation (as % of differentiated cells in culture) against time for both methods. In Prodigy-isolated cells, all lower cell density expansion conditions (500 CD34⁺ cells/ml, 2000 CD34⁺ cells/ml and 10000 CD34⁺ cells/ml) show a higher differentiation status than higher initial densities (40000 CD34⁺ cells/ml, 75000 CD34⁺ cells/ml and 100000 CD34⁺ cells/ml) through all the stages with more prominent advantage at earlier times (days 20-28). For manually-isolated CD34⁺ cells, a similar trend, though less prominent can be seen. Results are single data points or average of duplicates or quadruplicates. N=1 biological replicate.

FIG. 7—Influence of cell isolation method and expansion phase density on mature effector cells potency in complete medium. The potency of effector cells, determined as the % of killed target tumour cells after overnight co-culture with target tumour cells at a 1:1 effector:target ratio, is analysed via flow cytometry for Prodigy- and manually selected expansion conditions at day 35. The histograms show the % of target cells killing against density. Lower density conditions (500 CD34⁺ cells/ml, 10000 CD34⁺ cells/ml, and in particular 2000 CD34⁺ cells/ml) trigger higher killing compared to higher densities (75000 CD34⁺ cells/ml and 100000 CD34⁺ cells/ml), similarly for the two methods, but more prominent in Prodigy-isolated cells. Results are single data points or average of duplicates, each analysed in technical triplicates. N=1 biological replicate.

FIG. 8—Influence of seeding density on the expansion of CD34⁺ haematopoietic stem cells (HSCs) purified from fresh umbilical cord blood (UCB) in low density cytokine mix (LDC)-free expansion medium. A. Freshly isolated day 0 HSCs are cultured in LDC-free expansion medium at different, increasing densities and cell expansion is analysed via flow cytometry on days 6, 9 and 13-14. Similar cell densities are grouped: 500 CD34⁺ cells/ml, 2000 CD34⁺ cells/ml and 10000 CD34⁺ cells/ml: <10000 CD34⁺ cells/ml; 40000 CD34⁺ cells/ml, 75000 CD34⁺ cells/ml and 100000 CD34⁺ cells/ml: >40000 CD34⁺ cells/ml. The cell expansion is calculated as [cells/ml at day of measurement divided by CD34⁺ cells/ml at day 0] for every condition, is plotted against time. Cell seeding density lower than 10000 CD34⁺ cells/ml results in higher cell expansion at day 13. B. HSCs relative endpoint (day 13-14) expansion is calculated for each cell density on 100000 CD34⁺ cells/ml (set as =1) and plotted against the increasing density, where 500 CD34⁺ cells/ml is set as =1 (i.e., 1-20=500-10,000 CD34⁺ cells/ml and 80-200=40,000-100,000 CD34⁺ cells/ml). The histogram shows an inverse correlation between the two variables, indicating how the high expansion potential of low density seeded cells progressively reduces when cell density increases. Results are the mean (SD) of technical duplicates or triplicates for N=2 biological replicates; data are analysed using Student's t-test. ns=statistically non-significant difference; * p≤0.033.

FIG. 9—Influence of expansion phase density on CD34⁺ haematopoietic stem cells (HSCs)-derived progenitors' differentiation into mature effector cells in low density cytokine mix (LDC)-free differentiation medium. On day 13-14, cells are seeded for differentiation culture phase in LDC-free differentiation medium; all conditions are set at the same density of 1.5*10⁶cells/ml and cell differentiation phenotype is monitored via flow cytometry at days 20-21, 27-28 and 35-36. Similar cell densities are grouped: 500 CD34⁺ cells/ml, 2000 CD34⁺ cells/ml and 10000 CD34⁺ cells/ml: <10000 CD34⁺ cells/ml; 40000 CD34⁺ cells/ml, 75000 CD34⁺ cells/ml and 100000 CD34⁺ cells/ml: >40000 CD34⁺ cells/ml. The plot shows % of differentiated cells over time. No difference is observed between low (<10000 CD34⁺ cells/ml) and high (>40000 CD34⁺ cells/ml) densities. Results are the mean (SD) of single data points for N=4 biological replicates; data are analysed using Student's t-test. Any differences observed are statistically non-significant.

FIG. 10—Influence of expansion phase density on mature effector cells potency in low density cytokine mix (LDC)-free medium. The potency of effector cells, determined as the % of killed target tumour cells after overnight co-culture at a 1:1 effector:target ratio, is analysed via flow cytometry at day 35-36 for all expansion densities cultured in LDC-free medium. The plot shows the % of killed target cells for all densities, grouped as follows: 500 CD34⁺ cells/ml, 2000 CD34⁺ cells/ml and 10000 CD34⁺ cells/ml: <10000 CD34⁺ cells/ml; 40000 CD34⁺ cells/ml, 75000 CD34⁺ cells/ml and 100000 CD34⁺ cells/ml: >40000 CD34⁺ cells/ml. Killing potency is higher in low density conditions (<10000 CD34⁺ cells/ml) compared to higher density (>40000 CD34⁺ cells/ml) Results are the mean (SD) of technical triplicates for N=3 biological replicates; data are analysed using Student's t-test. * p≤0.033.

FIG. 11—Influence of seeding density on the expansion of CD34⁺ haematopoietic stem cells (HSCs) purified from fresh umbilical cord blood (UCB) in 2% human serum, 5x cytokines, low density cytokine mix (LDC)-free expansion medium. A. Freshly isolated day 0 HSCs are cultured in 2% human serum, 5x cytokines, low density cytokine mix (LDC)-free expansion medium at low (2000 CD34⁺ cells/ml) or high (40000 CD34⁺ cells/ml) density and cell expansion is analysed via flow cytometry on days 6, 9 and 14. The cell expansion measured over time, calculated as [cells/ml at day of measurement/CD34⁺ cells/ml at day 0] for every condition, is plotted against time. Seeding density of 2000 CD34⁺ cells/ml results in higher cell expansion, if compared to 40000 CD34⁺ cells/ml. B. HSCs relative endpoint (day 14) expansion is calculated for each cell density on 40000 CD34⁺ cells/ml (set as =1) and plotted against the increasing density, where 2000 CD34⁺ cells/ml is set as =1 (i.e., 1=2000 CD34⁺ cells/ml and 20=40,000 CD34⁺ cells/ml). The histogram shows an inverse correlation between the two variables, indicating how the high expansion potential of low density seeded cells progressively reduces when cell density increases. Results are the mean (SD) of single data points for N=2 biological replicates; data are analysed using Student's t-test. ns: statistically non-significant difference.

FIG. 12—Influence of expansion phase density on CD34⁺ haematopoietic stem cells (HSCs)-derived progenitors' differentiation into mature effector cells in 2% human serum, 5x cytokines, low density cytokine mix (LDC)-free differentiation medium. On day 14, 2000 CD34⁺ cells/ml and 40000 CD34⁺ cells/ml expansion conditions are seeded for differentiation culture phase in 2% human serum, 5x cytokines, low density cytokine mix (LDC)-free differentiation medium at the same density of 1.5*10⁶cells/ml and cell differentiation phenotype is monitored via flow cytometry at days 21, 27 and 35. The histogram shows the % of differentiated cells over time, with a slightly improved differentiation % for low density. Results are the mean (SD) of single data points for N=2 biological replicates; data are analysed using Student's t-test. Any differences observed are statistically non-significant.

FIG. 13—Influence of expansion phase density on mature effector cells potency in 2% human serum, 5x cytokines, low density cytokine mix (LDC)-free medium. The potency of effector cells, determined as the % of killed target tumour cells after overnight co-culture at a 1:1 effector:target ratio, is analysed via flow cytometry at day 35 for 2000 CD34⁺ cells/ml and 40000 CD34⁺ cells/ml expansion densities cultured in 2% human serum, 5x cytokines, low density cytokine mix (LDC)-free medium. The plot shows the % of killed target cells against cell density. Potency is slightly higher for 2000 CD34⁺ cells/ml. Results are the mean (SD) of technical triplicates for N=2 biological replicates; data are analysed Student's t-test. Any differences observed are statistically non-significant.

FIG. 14—Influence of seeding density on the expansion of CD34⁺ haematopoietic stem cells (HSCs) purified from fresh umbilical cord blood (UCB) in 2% human serum, 1x cytokines, low density cytokine mix (LDC)-free expansion medium. A. Freshly isolated day 0 HSCs are cultured in 2% human serum, 1x cytokines, low density cytokine mix (LDC)-free expansion medium at low (2000 CD34⁺ cells/ml) or high (40000 CD34⁺ cells/ml) density and cell expansion is analysed via flow cytometry on days 6, 9 and 14. The cell expansion, calculated as [cells/ml at day of measurement divided by CD34⁺ cells/ml at day 0] for every condition, is plotted against time. Seeding density of 2000 CD34⁺ cells/ml results in higher cell expansion, if compared to 40000 CD34⁺ cells/ml. B. HSCs relative endpoint (day 13-14) expansion is calculated for each cell density on 40000 CD34⁺ cells/ml (set as =1) and plotted against the increasing density, where 2000 CD34⁺ cells/ml is set as =1 (i.e., 1=2000 CD34⁺ cells/ml and 20=40,000 CD34⁺ cells/ml). The histogram shows an inverse correlation between the two variables, indicating how the high expansion potential of low density seeded cells progressively reduces when cell density increases. Results are the mean (SD) of single data points for N=2 biological replicates; data are analysed using Student's t-test. ns: statistically non-significant difference.

FIG. 15—Influence of expansion phase density on CD34⁺ haematopoietic stem cells (HSCs)-derived progenitors' differentiation into mature effector cells in 2% human serum, 1x cytokines, low density cytokine mix (LDC)-free differentiation medium. On day 14, 2000 CD34⁺ cells/ml and 40000 CD34⁺ cells/ml conditions are seeded for differentiation culture phase in 2% human serum, lx cytokines, low density cytokine mix (LDC)-free differentiation medium at the same density of 1.5*10⁶cells/ml and cell differentiation phenotype is monitored via flow cytometry at days 21, 27 and 35. The histogram shows % of differentiated cells over time, where low density shows a slight advantage at day 35. Results are the mean (SD) of single data points for N=2 biological replicates; data are analysed using Student's t-test. Any differences observed are statistically non-significant.

FIG. 16—Influence of expansion phase density on mature effector cells potency in 2% human serum, 1x cytokines, low density cytokine mix (LDC)-free medium. The potency of effector cells, determined as the % of killed target tumour cells after overnight co-culture at a 1:1 effector:target ratio, is analysed via flow cytometry at day 35 for 2000 CD34⁺ cells/ml and 40000 CD34⁺ cells/ml expansion densities cultured in 2% human serum, 1x cytokines, low density cytokine mix (LDC)-free medium. The plot shows the % of killed target cells against cell density. Potency is higher for 2000 CD34⁺ cells/ml. Results of technical triplicates for N=1 biological replicate is shown.

FIG. 17—Influence of day 0 seeding density on the day 35 endpoint expansion of Effector Cells cultured in complete expansion medium. Endpoint (day 35-36) expansion of effector cells is measured and plotted against day 0 haematopoietic stem cells seeding density. Low cell densities are grouped: 500 CD34⁺ cells/ml and 2000 CD34⁺ cells/ml: <3000 CD34⁺ cells/ml; other cell densities tested: 10000 CD34⁺ cells/ml; 40000 CD34⁺ cells/ml; 75000 CD34⁺ cells/ml and 100000 CD34⁺ cells/ml. The cell expansion is calculated as [cells/ml at day of measurement divided by CD34⁺ cells/ml at day 0] for every condition. Low density-seeded cells, up to 10000 CD34⁺ cells/ml, and in particular <3000 CD34⁺ cells/ml, show that the beneficial effect on cell expansion is maintained through the whole culture, even if all cells are seeded at the same density for the differentiation phase. Results are the mean (SD) of technical duplicates or triplicates for N=4 biological replicates;

data are analysed using 1-way ANOVA. Any differences observed are statistically non-significant.

### DETAILED DESCRIPTION OF THE INVENTION

In a first embodiment, the invention provides a method for producing a collection of stem cells, progenitor cells, and/or NK cells, said method comprising the step of (i) initiating a cell culture from a sample comprising CD34⁺ human stem cells and culturing the cells for at least 7 days, preferably at least 9 days, in a basic culture medium comprising interleukin-7 (IL-7) and stem cell factor (SCF) and one or more of flt-3Ligand (FLT-3L) and thrombopoietin (TPO), characterized in that the cell culture is initiated at a cell density of 12,000 CD34⁺ cells/ml or less. Preferably the cell culture is initiated at a cell density of between 500 and 10,000 CD34⁺ cells/ml, more preferably at a cell density of between 1,000 and 8,000 CD34⁺ cells /ml, most preferably between 2,000 and 6,000 CD34⁺ cells/ml. In a preferred embodiment, the basic culture medium further comprises two or more of GM-CSF, G-CSF and IL-6.

Preferably step (i) of a method according to the invention, comprises culturing the cells for 7-10 days, more preferably 9 days in said culture medium.

Preferably, the method further comprises a step (ii) of culturing the cells obtained in step (i) for at least 4 days, more preferably at least 5 days, more preferably between 5-7 days, more preferably 5 days, in a medium comprising IL-15 and IL-7 and one or more of SCF or FLT-3L. Preferably the medium in step (ii) further comprises two or more of GM-CSF, G-CSF and IL-6.

The culture of step (ii) of a method of the invention is preferably performed in a disposable bag for culturing mammalian cells, preferably under static conditions. The disposable bag allows for the culture to be performed in a closed system that is necessary for clinical applications. Step ii is preferably performed under static conditions. It has been found that static conditions are preferred in this stage as this allows good yields of cultured stem cells and progenitor cells or both at the end of step ii. Without being bound by theory it is thought that the static conditions allow the cells to settle and associated with the wall of the disposable bag and to associate with neighbouring cells and that this association favourably affects the yield of the desired cells.

Typically and preferably, the addition of IL-15 that is present in step (ii) and preferably absent in step (i) is effectuated by aspirating half of the medium used in step (i) and addition of new medium comprising twice the amount of cytokines and growth factors as indicated above. The cells are preferably not washed, centrifuged, and/or concentrated or diluted during transition from step (i) to step (ii).

In one preferred embodiment, the method further comprises the step of (iii) culturing cells obtained in step (ii), preferably while the culture medium is continuously mixed during culture, for at least 13 days, preferably between 14 - 28 days, more preferably between 19-23 days, most preferably for 21 days, at a cell density of between 0.5×10⁶cells/ml-20×10⁶ cells/ml, preferably between 0.75×10⁶cells/ml-10×10⁶ cells/ml, more preferably between 1×10⁶ cells/ml and 5×10⁶ cells/ml, more preferably between 1.2×10⁶cells/ml and 4×10⁶ cells/ml, most preferably between 1.5×10⁶cells/ml and 3×10⁶cells/ml in a culture medium comprising three or more of SCF, IL-7, IL-15 and IL-2, thereby obtaining a collection of cultured cells containing a plurality of NK cells. In one preferred embodiment, the culture medium used in step (iii) of a method according to the invention further comprises two or more of GM-CSF, G-CSF, and IL-6. The culture of step iii of a method of the invention is preferably performed while the culture medium is mixed during culture, to enhance gas-exchange and to reduce the adherence of cells to a solid surface, which increases the purity of the obtained NK cells. In a preferred embodiment of a method of the invention the cells obtained in step (iii), are harvested. The harvested cells can be used directly for adoptive cell transfer purposes. Such adoptive cell transfer is preferably performed for the treatment of any kind of human disease preferably all malignant diseases such as tumours, cancer, leukemias as well as all viral diseases, also in solid transplant rejection situations and autoimmune diseases and loss of pregnancy.

In a preferred embodiment the harvested cells are washed in a closed system such that culture medium components are diluted at least 500 fold and are replaced by a solution that is compatible with human administration comprising human serum albumin. It is preferred that said solution with which the cells are washed does not contain human serum. It is preferred that the human serum albumin present in the solution is derived from a batch comprising essentially pure human serum albumin. In a preferred embodiment said human serum albumin is recombinantly produced human serum albumin. In a preferred embodiment said solution comprises between 0,3% and 10% human serum albumin. Preferably said solution comprises between 0,5 and 5% humans serum albumin. It has been observed that cells treated in the above way and are collected in the solution that is compatible with human administration and that comprises human serum albumin can be stored for a prolonged period of time under these conditions without detrimental loss of viability and/or activity. The solution wherein the cells are stored is further also referred to as “storage solution”. The storage solution preferably comprises less than 0.1% human serum, preferably said storage solution does not comprise human serum. In a preferred embodiment said storage solution comprises human serum derived from a batch comprising essentially pure human serum albumin. In a preferred embodiment said human serum albumin is recombinantly produced human serum albumin. In a preferred embodiment said storage solution comprises between 0,3% and 10% human serum albumin. Preferably said storage solution comprises between 0,5 and 5% humans serum albumin. Preferred solutions compatible with human administration are preferably PBS or physiological salt solutions. The PBS or physiological salt solution may contain one or more additives. In one embodiment the additive is human serum albumin. In a preferred embodiment the compatible solution is physiological salt solution. Harvested cells are preferably stored for at least one day at a temperature of between room temperature and 0° C., preferably said harvested cells are stored for 1, 2 or 3 days at said temperature. Preferably said solution that is compatible with human administration is a physiological salt solution. The physiological salt solution is typically though not necessarily 0,9% NaCl. In one embodiment, harvested and/or stored cells are divided into at least 5 portions and stored at a temperature below —70° C. Preferably, said solution comprises up to 75% of Cryostor® CS 10 cryopreservation media, which can be ordered at Sigma-Aldrich. Cryostor CS 10 comprises 10% DMSO, end concentration DMSO in said solution is thus 7.5%.

Typically, one infusion bag, comprising one dosage of cells, comprises 500-1000×10⁶ cells, in 25 ml of solution as described above, preferably comprising 75% (v/v) Cryostor CS 10 and 25% (v/v) NaCl 0.9%.

It has been observed that in contrast to starting with a higher cell density at day 0, e.g. above 10,000 CD34⁺ cells/ml, like 40,000 CD34⁺ cells/ml, a method according to the invention starting with an initial cell density of between 500-10,000 CD34⁺ cells/ml surprisingly results in a much higher expansion of the haematopoietic stem cells and/or NK progenitor cells. This higher expansion is already seen after step (i) and continues to be higher up to and including step (iii). In a working example, the method resulted in a relative cell expansion between 10 to 16 times higher if cells are seeded at only 500-2000 CD34⁺ cells/ml, compared to 100,000 CD34⁺ cells/ml. On day 12-15, between 150- and 700-fold expansions were reached when cells were seeded at an initial density of between 500-10,000 cells /ml, whereas at higher densities, the fold expansion was below 100. Further, and even more surprising, the increased expansion even carried on after days 8-9 in the cultures initiated at lower density, whereas in those with higher initiated cell density showed limited expansion. This was happening during the differentiation phase, where the cells were set at the same density (1-2×10⁶ cells/ml) at day 12-15, whereafter their expansion during differentiation phase was followed. In a preferred embodiment, a method according to the invention is provided, wherein the method results in an at least 150-fold expansion of cells, preferably at least 200-fold, more preferably at least 300-fold, most preferably at least 500-fold at day 12. Preferably, a method according to the invention results in an at least 150-fold expansion of cells, preferably at least 200-fold, more preferably at least 400-fold, most preferably at least 800-fold at day 15.

The CD34⁺ stem cells may be sourced from, e.g., cord blood, placenta, peripheral blood, bone marrow, and the like. Preferably, the sample comprising CD34⁺ human stem cells is obtained from human cord blood. Methods for obtaining CD34⁺ stem cells from these sources, either using manual selection or fully automated are known by the skilled person and, e.g., described in the Examples and in various publications (Christopher Y Park et al, Nature Protocols. Vol. 3, Issue 12. (December 2008) p1932-1940; Avecilla ST et al, Transfusion. 2016 May;56(5):1008-12 ; Hümmer C et al, J Transl Med. 2016 March 16;14:76). It is preferred that the sample comprising CD34⁺ human stem cells is obtained by selecting CD34⁺ human stem cells (HSC) through a fully automated closed system, such as for instance a clinical GMP-compliant CliniMACS Prodigy® by Miltenyi Biotec GmbH, and collected directly in culture bags for further culturing in step (i) of a method according to the invention. The method of the invention enables such procedure because, typically, the CD34⁺ HSC obtained by a fully automated closed system are present, after selection, in a cell density as is, or higher than used for initiating a CD34⁺ cell culture in a method according to the invention. Prior to the present invention, the thus obtained CD34⁺ HSC fraction had to be concentrated, e.g., through centrifugation and resuspension in a lower volume of medium in order to achieve at least 50,000 -100,000 CD34⁺ cells/ml. Thus, for the first time, a closed system can be used that fully automatically collects and cultures CD34⁺ human stem cells, without any steps outside of the closed system, thereby preventing contamination with, e.g., air-born micro-organisms and reducing hands-on time.

In one aspect, the invention thus provides a method for producing a collection of stem cells, progenitor cells, and/or NK cells, said method comprising the steps of
selecting CD34⁺ HSC through a fully automated closed system;
collecting and culturing the thus selected CD34⁺ HSC at a cell density of not more than 10,000 CD34⁺ cells/ml, preferably between 500 and 10,000 CD34⁺ cells/ml, more preferably between 1,000 and 8,000 CD34⁺ cells/ml, more preferably between 2,000 and 6,000 CD34⁺ cells/ml from a sample comprising CD34⁺ human stem cells and culturing the cells for at least 7 days in a basic culture medium comprising stem cell factor (SCF) and interleukin-7 (IL-7), and one or more of flt-3Ligand (FLT-3L)and thrombopoietin (TPO), wherein the collecting step is preferably within the fully automated closed system. The method can further be extended by performing step (ii), or step (ii) and step (iii), as described previously. With the term “within the fully automated closed system” in this context is meant that the cells are collected, without manual interference, into suitable culturing means, such as a transfer bag, culture bag or bioreactor. The cells thereby preferably do not leave the closed system. The cells are, preferably, not concentrated between the collection and culturing step. If necessary, the cells may be diluted with fresh medium after the collecting step using means of the closed system for adding medium to the culture in order to obtain lower cell density cultures. Such means can, e.g., be sterile welding a 3-way valve and tubing adding medium by gravity (flow from higher bag to lower bag) or by use of a syringe (take medium from medium bag by pulling, push into culture bag after having changed the valve pathway..

Nevertheless, if handled properly, the CD34⁺ human stem cells may also be obtained manually. In another preferred embodiment, therefore, a method according to the invention is provided, wherein the sample comprising CD34⁺ stem cells from human postembryonic tissue is obtained by selecting CD34⁺ human stem cells (HSC) through manual column separation, preferably using CD34 positive selection, e.g., as described in the Examples.

Preferably, the growth factors and cytokines mentioned above are, independently from one another and if present, used in the following concentrations: SCF, FLT-3L, TPO, IL-7, and IL-15 at a concentration between 2 ng/ml and 200 ng/ml, preferably between 4 ng/ml and 100 ng/ml, more preferably between 10 and 50 ng/ml, most preferably at a concentration of about 20 ng/ml; IL-2 at a concentration of between 100-10,000 U/ml, preferably between 200-5,000 U/ml, more preferably between 500-2,000 U/ml, most preferably at a concentration of about 1,000 U/ml; IL-6 at a concentration of between 5-500 pg/ml, preferably between 20-200 pg/ml, more preferably between 40-100 pg/ml, most preferably at a concentration of about 50 pg/ml; GM-CSF at a concentration of between 1-100 pg/ml, preferably between 2-50 pg/ml, more preferably between 5-25 pg/ml, most preferably at a concentration of about 10 pg/ml; and G-CSF at a concentration of between 25-2,500 pg/ml, more preferably between 100-1000 pg/ml, more preferably between 200-500 pg/ml, most preferably at a concentration of about 250 pg/ml.

Thus, in a preferred embodiment, a method according to the invention is provided, wherein, independently from one another, and if present, SCF is present at a concentration of between 2 ng/ml and 200 ng/ml, preferably between 4 ng/ml and 100 ng/ml, more preferably between 10 and 50 ng/ml, most preferably at a concentration of about 20 ng/ml; FLT-3L is present at concentration of between 2 ng/ml and 200 ng/ml, preferably between 4 ng/ml and 100 ng/ml, more preferably between 10 and 50 ng/ml, most preferably at a concentration of about 20 ng/ml; TPO is present at concentration of between 2 ng/ml and 200 ng/ml, preferably between 4 ng/ml and 100 ng/ml, more preferably between 10 and 50 ng/ml, most preferably at a concentration of about 20 ng/ml; IL-7 is present at a concentration of between 2 ng/ml and 200 ng/ml, preferably between 4 ng/ml and 100 ng/ml, more preferably between 10 and 50 ng/ml, most preferably at a concentration of about 20 ng/ml; IL-15 is present at a concentration of between 2 ng/ml and 200 ng/ml, preferably between 4 ng/ml and 100 ng/ml, more preferably between 10 and 50 ng/ml, most preferably at a concentration of about 20 ng/ml; IL-2 is present at a concentration of between 100-10,000 U/ml, preferably between 200-5,000 U/ml, more preferably between 500-2,000 U/ml, most preferably at a concentration of about 1,000 U/ml; IL-6 is present at a concentration of between 5-500 pg/ml, preferably between 20-200 pg/ml, more preferably between 40-100 pg/ml, most preferably at a concentration of about 50 pg/ml; GM-CSF is present at a concentration of between 1-100 pg/ml, preferably between 2-50 pg/ml, more preferably between 5-25 pg/ml, most preferably at a concentration of about 10 pg/ml; and G-CSF is present at a concentration of between 25-2,500 pg/ml, more preferably between 100-1000 pg/ml, more preferably between 200-500 pg/ml, most preferably at a concentration of about 250 pg/ml.

With “about” in this context is meant 20%, preferably 10% less or more. Thus about 250 pg/ml means between 200-300 pg/ml, preferably between 225-275 pg/ml. Such variation is typically the result of pipetting and other errors.

The invention surprisingly shows that starting with a concentration as described, i.e., below or at 12,000 CD34⁺ cells/ml, preferably between 500-10,000, more preferably between 1,000 and 8,000, most preferably between 2,000 and 6,000 CD34⁺ cells/ml, not only results in higher expansion but also in higher differentiation into NK cells. In one preferred embodiment, a method according to the invention is provided, wherein the NK cells obtained after step iii comprise at least 50%, preferably at least 60%, more preferably at least 75%, most preferably at least 80% fully differentiated NK cells after 28 days of culture. Preferably, the NK cells obtained after step iii comprise at least 75%, more preferably at least 80%, more preferably at least 85%, most preferably at least 90% fully differentiated NK cells after 35 days of culture. With fully differentiated in this context is meant that the cell is CD56+ and CD3-. Thus with, e.g., at least 80% fully differentiated NK cells is meant that at least 80% of the cells obtained from a method according to the invention are CD56+ and CD3−.

It has further been observed that a method according to the invention results in NK cells with excellent cytotoxic properties. In a working example, a method according to the invention, wherein the CD34⁺ HSC were seeded in an initial concentration of 2,000 CD34⁺ cells/ml resulted in NK cells that were able to kill on average 49% of K562 cells in a 1 effector cells to 1 target cell ratio. If the CD34⁺ HSC were seeded in conventional density (about 100,000 CD34⁺ cells/ml), only about 29% cell killing was observed. In a preferred embodiment, therefore, a method according to the invention is provided, wherein the NK cells obtained are able to kill at least 30%, more preferably at least 40%, most preferably at least 45% of their target cells, when measured in a cell cytotoxicity assay against K562 cells in a 1 effector cells to 1 target cell ratio. Such cytotoxicity assay is known in the art and is described in the Examples. In particular, in this context reference is made to the assay as described in Example 1.

Now that the invention provides a method as described above, the invention also provides a collection of NK cells obtained by a method according to the invention. Preferably such collection is obtained from a method wherein the cells are cultured for at least 7 days, preferably at least 9 days in step (i), at least 4 days, preferably at least 5 days in step (ii) and at least 13 days, preferably at least 21 days, in step (iii). It is in particular preferred, from a regulatory perspective, but also from a perspective of efficiency, that a composition for use according to the invention is obtained from a single donor. Even more preferred is that a single donor provides more than one treatment dose, such that large scale batches can be produced, be cleared or certified, and used off-the-shelf at the moment a random individual must be treated with a composition for use according to the invention. With “off-the-shelf” as used herein is meant that such composition is prepared and stored for direct usage when needed. In particular a composition that is available “off-the-shelf” is not generated for one specific recipient but in general can be used for different recipients at different time points. The collection as defined by the invention can for instance be frozen and, when needed, thawed and used as defined by the invention. A collection as defined by the invention enables large scale production of GMP generated immune effector cells that can theoretically be provided within minutes when needed for any random recipient.

Preferably such collection of NK cells comprises at least 10,000,000,000 (10¹⁰) cells from a single donor, more preferably such collection of NK cells comprises at least 20,000,000,000 (2×10¹⁰) cells, most preferably at least 25,000,000,000 (2.5×10¹⁰) cells from a single donor. A collection according to the invention preferably comprises more than 80%, preferably more than 85%, most preferably more than 90%, CD56 positive, CD3 negative cells, preferably said cells are negative for CD117 and CD34. In a preferred embodiment, a collection according to the invention is provided, wherein that part of the collection that is to be administered in one treatment comprises less than 1×10⁷ CD3 positive cells, more preferably less than 1×10⁶ CD3 positive cells, more preferably less than 1×10⁵ CD3 positive cells. In a preferred embodiment, the % of CD3 positive cells in relation to the number of total cells present in the composition does not exceed 1%, more preferably 0.1%, and most preferably it does not exceed 0.01% in relation to the total number of cells present in the composition. In a preferred embodiment, the % of CD19 positive cells in relation to the number of total cells present in the composition does not exceed 1%, more preferably 0.1%, and most preferably it does not exceed 0.01% in relation to the total number of cells present in the composition.

In a preferred embodiment said plurality of NK-cells or NK progenitor cells or both comprise at least 70%, more preferably at least 75%, more preferably at least 80%, most preferably at least 85% viable NK-cells or NK progenitor cells or both, preferably as determined by 7AAD exclusion.

Also provided is a pharmaceutical composition comprising a collection of NK cells according to the invention. Such pharmaceutical composition may be distributed in parts, for multiple injections/infusions in patients in need thereof. For instance, starting from 10¹⁰ cells from a single donor, 9 doses of 1×10⁹ CD56+CD3-NK cells may be prepared, for instance for three patients, each receiving three consecutive dosages. If 2.5×10¹⁰ cells are obtained, 8 patients may be treated with each 3 doses of 1×10⁹ CD56+CD3-NK cells. Typically a patient is to receive at least three doses of about 1×10⁹ cells, of which more than 70% are CD56+CD3⁻, but the skilled person, doing routine pharmacokinetic and -dynamic studies will be able to determine a therapeutically effective regimen with less or more dosages in combination with, e.g., more or less cells per dosage. Before the present invention, and without using feeder cells or chemical compounds, it was only possible to prepare about 3 doses of 1×10⁹ cells, thereby only preparing dosages for a single patient, from a high-output cord blood unit. Furthermore, the present invention makes it possible to also use non-high-output cord blood units, making up 80% of all cord blood units available, which had been discarded previously. In one preferred embodiment, a method according to the invention is provided, wherein no feeder cells are added to the cell culture. In this respect it is to be noted that with the expression “no feeder cells are added” is meant that such cells are not intentionally and additionally added to the cell culture, i.e., not as part of the sample comprising CD34⁺ human stem cells from which the cell culture is initiated.

As a pharmaceutical composition according to the invention comprises a sufficient amount and purity of NK cells with excellent cytotoxic properties, the invention further provides such pharmaceutical composition for use in medicine, preferably for use in immunotherapy. Preferably the pharmaceutical composition according to the invention is for use in the treatment of tumours and haematological malignancies.

The composition of the invention can be administered through any acceptable method, provided the immune effector cells are able to reach their target in the individual. It is for instance possible to administer the composition of the invention via the intravenous route or via a topical route, including but not limited to the ocular, dermal, pulmonary, buccal and intranasal route. With topical route, as used herein, is also meant any direct local administration such as for instance in the bone marrow, but also directly injected in, e.g., a solid tumour. In particular cases, e.g. if the immunotherapy is aimed at an effect on the mucosal layer of the gastrointestinal tract, the oral route can be used.

Preferably, a composition for a use according to the invention is provided, wherein the composition is administered by intravenous route or by a topical route or by oral route or by any combination of the three routes. With the term “topical” as used herein is meant, that the immune effector cells are applied locally, preferably at the site of tumour, which can be localized in any anatomical site, more specifically the tumour can be localized in the bone marrow or any other organ. The composition for use according to the invention can be administered once, but if deemed necessary, the composition may be administered multiple times. These can be multiple times a day, a week or even a month. It is also possible to first await the clinical result of a first administration, e.g. an infusion and, if deemed necessary, give a second administration if the composition is not effective, and even a third, a fourth, and so on.

As already elaborated before, a composition for use according to the invention is especially useful in immunotherapy for the treatment of a tumour. Without being bound to therapy, the HLA mismatched immune effector cell is thought to kill tumour cells through secretory lysosome exocytosis after recognizing its target. Target cell recognition induces the formation of a lytic immunological synapse between the immune effector cell and its target. The polarized exocytosis of secretory lysosomes is then activated and these organelles release their cytotoxic contents at the lytic synapse, specifically killing the target cell. The composition for use according to the invention for use in the treatment of a tumour is useful for both hematopoietic or lymphoid tumours and solid tumours. In a preferred embodiment, a composition according to the invention is provided, wherein the immune effector cell is able to kill a tumour cell through secretory lysosome exocytosis.

In one preferred embodiment, a composition for a use according to the invention for the treatment of a tumour is provided, wherein the tumour is a hematopoietic or lymphoid tumour or wherein tumour is a solid tumour.

With the term “haematological”, “haematopoietic” or “lymphoid” tumour is meant, that these are tumours of the hematopoietic and lymphoid tissues. Hematopoietic and lymphoid malignancies are tumours that affect the blood, bone marrow, lymph, and lymphatic system.

The present invention shows exemplary results for the effectiveness of a composition of the invention for use in both, the treatment of a hematopoietic and of solid tumours.

In those cases that the tumour is a hematopoietic or lymphoid tumour, a composition for use according to the invention is provided, wherein the tumour is one or more of leukaemia, lymphoma, myelodysplastic syndrome or myeloma, preferably a leukaemia, lymphoma or myeloma selected from acute myelogenous leukaemia (AML), chronic myelogenous leukaemia (CML), acute T cell leukaemia, acute lymphoblastic leukaemia (ALL), chronic lymphocytic leukaemia (CLL), acute monocytic leukaemia (AMoL), mantle cells lymphoma (MCL), histiocytic lymphoma or multiple myeloma, preferably AML.

In those cases that the tumour is a solid tumour, a composition for use according to the invention is provided, wherein the tumour is one of malignant neoplasms or metastatic induced secondary tumours of adenocarcinoma, squamous cell carcinoma, adenosquamous carcinoma anaplastic carcinoma, large cell carcinoma or small cell carcinoma, hepatocellular carcinoma, hepatoblastoma, colon adenocarcinoma, renal cell carcinoma, renal cell adenocarcinoma, colorectal carcinoma, colorectal adenocarcinoma, glioblastoma, glioma, head and neck cancer, lung cancer, breast cancer, Merkel cell cancer, rhabdomyosarcoma, malignant melanoma, epidermoid carcinoma, lung carcinoma, renal carcinoma, kidney adenocarcinoma, breast carcinoma, breast adenocarcinoma, breast ductal carcinoma, non-small cell lung cancer, ovarian cancer, oral cancer, anal cancer, skin cancer, Ewing sarcoma, stomach cancer, urethral cancer, uterine cancer, uterine sarcoma, vaginal cancer, vulvar cancer, Wilms tumour, Waldenstrom macroglobulinemia, pancreas carcinoma, pancreas adenocarcinoma, cervix carcinoma, squamous cell carcinoma, medulloblastoma, prostate carcinoma, colon carcinoma, colon adenocarcinoma, transitional cell carcinoma, osteosarcoma, ductal carcinoma, large cell lung carcinoma, small cell lung carcinoma, ovary adenocarcinoma, ovary teratocarcinoma, bladder papilloma, neuroblastoma, glioblastoma multiforma, glioblastoma astrocytoma, epithelioid carcinoma, melanoma or retinoblastoma.

In a preferred embodiment, a composition for use according to the invention is provided, wherein the solid tumour is selected from malignant neoplasms or metastatic induced secondary tumours of cervical cancers selected from adenocarcinoma, squamous cell carcinoma, adenosquamous carcinoma, cervix carcinoma, small cell carcinoma, and melanoma. In another preferred embodiment, a composition for use according to the invention is provided, wherein the solid tumour is selected from malignant neoplasms or metastatic induced secondary tumours of colorectal cancers selected from adenocarcinoma, squamous cell carcinoma, colon adenocarcinoma, colorectal carcinoma, colorectal adenocarcinoma, colon carcinoma, and melanoma.

The composition of the invention has several advantages with respect to treatment options known to date. The composition of the invention is beneficial independent of HPV types, tumour histology, tumour EGFR expression and KRAS status. In addition to it, the immune effector cell of the invention also overcomes HLA-E, HLA-G and (IDO) inhibition, thus resulting in enhanced anti-tumour effects against tumours, especially against cervical cancers and colorectal cancers.

The term “Epidermal growth factor receptor” or EGFR as it is commonly described, refers to a cell surface protein widely expressed in almost all healthy tissues. The EGFR protein is encoded by transmembrane glycoprotein and is a member of the protein kinase family. Overexpression of EGFR and mutations in its downstream signalling pathway has been associated with bad prognosis in several solid tumours like colon, lung and cervix.

The term Kirsten rat sarcoma viral oncogene (KRAS) refers to the gene actively involved in regulating normal tissue signalling, part of EGFR downstream signalling pathway. However, mutations in the KRAS gene has been reported in tumour cells in solid tumours of colon, rectum and lungs. These activating mutations occurring in more than 50% of colorectal cancer patient helps tumour cells to evade EGFR targeting drugs like cetuximab and panitumumab.

The term “human papilloma virus (HPV) as used herein refers to the group of viruses which causes cervical cancer in women. HPV virus affects the skin and moist membranes surrounding mouth, throat, vulva, cervix and vagina. HPV infection causes abnormal cell changes that leads to cancer in the cervix.

The term Indoleamine 2,3 dioxygenase (IDO) as used herein refers to an enzyme which acts as a catalyst in degrading amino acids L-tryptophan to N-formylkynurenine. Overexpression of IDO commonly reported in solid tumours of prostate, gastric, ovarian, cervix and colon, enables tumour cells to evade killing by cytotoxic T cells and NK cells.

For those jurisdictions that allow claims on medical treatment, the following embodiments are also provided by the invention. Each and every embodiment listed below may be combined with each other and/or with any embodiment described above.

Method for treating an individual in need of immunotherapy, the method comprising administering to the individual a composition according to the invention, wherein the composition comprises a therapeutically effective amount of CD56+CD3- cells.

Method for treating an individual in need of immunotherapy according to the invention, wherein the immunotherapy is for the treatment of a tumour.

Method for treating an individual in need of immunotherapy according to the invention, the method further comprising administering, prior to the administration of the composition according to the invention, cyclophosphamide and/or fludarabine to said individual, characterized in that the cyclophosphamide is dosed on 2, 3, 4 or 5 subsequent days at a total dose of 400 - 10000 mg/m2, preferably 800-8000, more preferably 1600-6000, more preferably 2000-4000, most preferably about 3600 mg/m2, and/or the fludarabine is dosed on 2, 3, 4, or 5 subsequent days at a total dose of 1-1000, preferably 10-500, more preferably 50-250, most preferably about 120 mg/m2.

Method for treating an individual in need of immunotherapy according to the invention, wherein the composition to be administered in one treatment comprises at least 5×10⁸ CD34⁺ cells, more preferably at least 1×10⁹ CD34⁺ cells.

Method for treating an individual in need of immunotherapy according to the invention, wherein the composition to be administered in one treatment comprises not more than 1×10¹⁰ CD34⁺ cells.

Method for treating an individual in need of immunotherapy according to the invention, wherein the composition to be administered in one treatment comprises less than 1×10′ CD3 positive cells.

Method for treating an individual in need of immunotherapy according to the invention, wherein composition to be administered in one treatment comprises less than 1×10′ CD19 positive cells.

Method for treating an individual in need of immunotherapy according to the invention, wherein the composition is administered by intravenous route.

Method for treating an individual in need of immunotherapy according to the invention, wherein the composition is administered by a topical route.

Method for treating an individual in need of immunotherapy according to the invention, wherein the tumour is a hematopoietic or lymphoid tumour or wherein tumour is a solid tumour.

Method for treating an individual in need of immunotherapy according to the invention, wherein the tumour is a hematopoietic or lymphoid tumour, selected from leukaemia, lymphoma, myelodysplastic syndrome or myeloma, preferably a leukaemia, lymphoma or myeloma selected from acute myelogenous leukaemia (AML), chronic myelogenous leukaemia (CML), acute T cell leukaemia, acute lymphoblastic leukaemia (ALL), chronic lymphocytic leukaemia (CLL), acute monocytic leukaemia (AMoL), mantle cells lymphoma (MCL), histiocytic lymphoma, multiple myeloma, any others?.

Method for treating an individual in need of immunotherapy according to the invention, wherein the leukaemia is AML.

Method for treating an individual in need of immunotherapy according to the invention, wherein the tumour is a solid tumour, selected from malignant neoplasms or mestastatic induced secondary tumours of adenocarcinoma, squamous cell carcinoma, adenosquamous carcinoma anaplastic carcinoma, large cell carcinoma or small cell carcinoma, hepatocellular carcinoma, hepatoblastoma, colon adenocarcinoma, renal cell carcinoma, renal cell adenocarcinoma, colorectal carcinoma, colorectal adenocarcinoma, glioblastoma, glioma, head and neck cancer, lung cancer, breast cancer, Merkel cell cancer, rhabdomyosarcoma, malignant melanoma, epidermoid carcinoma, lung carcinoma, renal carcinoma, kidney adenocarcinoma, breast carcinoma, breast adenocarcinoma, breast ductal carcinoma, non-small cell lung cancer, ovarian cancer, oral cancer, anal cancer, skin cancer, Ewing sarcoma, stomach cancer, urethral cancer, uterine cancer, uterine sarcoma, vaginal cancer, vulvar cancer, Wilms tumor, Waldenstrom macroglobulinemia, pancreas carcinoma, pancreas adenocarcinoma, cervix carcinoma, squamous cell carcinoma, medulloblastoma, prostate carcinoma, colon carcinoma, colon adenocarcinoma, transitional cell carcinoma, osteosarcoma, ductal carcinoma, large cell lung carcinoma, small cell lung carcinoma, ovary adenocarcinoma, ovary teratocarcinoma, bladder papilloma, neuroblastoma, glioblastoma multiforma, glioblastoma astrocytoma, epithelioid carcinoma, melanoma and retinoblastoma.

In a preferred embodiment, a method according to the invention is provided, wherein the solid tumour is selected from malignant neoplasms or metastatic induced secondary tumors of cervical cancers selected from adenocarcinoma, squamous cell carcinoma, adenosquamous carcinoma, cervix carcinoma, small cell carcinoma, and melanoma.

In another preferred embodiment, a method according to the invention is provided, wherein the solid tumour is selected from malignant neoplasms or metastatic induced secondary tumors of colorectal cancers selected from adenocarcinoma, squamous cell carcinoma, colon adenocarcinoma, colorectal carcinoma, colorectal adenocarcinoma, colon carcinoma, and melanoma.

The Invention is further exemplified in the following non-limiting Examples.

### EXAM P LES

### Example 1

Introduction

Human CD34⁺ haematopoietic stem cells (HSCs) isolated from fresh umbilical cord blood (UCB) can give rise to effector cells able to kill target tumour cells via a multi-step in vitro process based on an initial expansion phase (days 0-14), followed by an effector cell-specific differentiation phase (days 14-35/42). Optimization of the expansion and differentiation conditions is useful to generate large batches of highly differentiated effector cells. Here, the influence of day 0 CD34⁺ HSCs cell culture density on the expansion, differentiation and potency of effector cells to kill target cells is analysed in complete culture medium. UCB-derived CD34⁺ HSCs are seeded for expansion phase at 6 or more different, increasing densities (expressed as cells/ml), and monitored between days 0 and 12-14, when intermediate (days 5-6 and 8-9) and final cell expansion is determined via flow cytometry. At day 12-15, all conditions are set at the same density for the differentiation phase, to determine the effect of the initial expansion density on the subsequent differentiation. At days 20-21, 27-28 and 35-36, effector cells differentiation is monitored via flow cytometry. At day 35-36, endpoint effector cells' potency is analysed via an in vitro assay where effector cells are co-cultured overnight with target tumour cells and killing of targets is assessed via flow cytometry.

Materials and Methods

Tumour Cell Lines

Tumour cell lines, used in the effector cell potency assay, are cultured in IMDM medium (Iscove's modified Dulbecco's medium, Lonza, Maastricht, NL) containing 100 U/ml penicillin and 100 U/ml streptomycin (Lonza), 2 mM L-Glutamine (Lonza) and 10% foetal bovine serum (FBS, Fisher Scientific, Landsmeer, NL). Cell cultures are passaged every 5 days and maintained at 37° C., 95% humidity, 5% CO₂ in a cell incubator.

CD34⁺ Haematopoietic Stem Cells (HSCs) Isolation from Umbilical Cord Blood (UCB)

CD34⁺ HSCs are isolated from fresh umbilical cord blood units (supplied by Anthony Nolan, London UK) with two methods, an automatic closed system or a manual open system. Fully automatic selection is performed by using the closed immunomagnetic CliniMACS Prodigy® LP-34 System (Miltenyi Biotec GmbH, Bergisch Gladbach, Germany). The CliniMACS Prodigy TS310 tubing set is installed on the instrument and the CliniMACS PBS/EDTA (Phosphate Buffer Saline/Ethylenediaminetetraacetic acid) buffers are connected. After priming of the instrument, the fresh umbilical cord blood bag is connected and the CliniMACS CD34 Reagents for labelling of CD34⁺ cells are added. The magnetically labelled CD34⁺ cells are retained in the separation column (in which strong magnetic fields are generated) for washing steps, then eluted in 80 ml of cell culture medium after removing the magnetic field. All steps are performed according to the CliniMACS Prodigy° LP-34 System user manual.

Manual selection is performed by pipetting fresh blood diluted 1:3 with PBS on top of a Ficoll-Paque (GE Healthcare, Hoevelaken, NL) layer in a sterile tube, then centrifuging at 900 g for 30 min at 20° C., with brake turned off. The separated mononuclear cell layer is transferred to a sterile tube using a pipette; cells are then washed twice with PBS (Lonza). Cells are counted via flow cytometry and CD34⁺ cells are labelled with the CD34 MicroBead Kit (Miltenyi Biotec); after labelling, the cells are loaded on a LS Column placed on a MultiStand (both from Miltenyi Biotec) to apply a magnetic field, unlabelled cells are washed, then the CD34⁺ HSCs are eluted in 15 ml of FACS buffer composed of 1x PBS supplemented with 0.5% Albumin (Sanquin, Amsterdam, NL) and 2 mM EDTA pH 8.0 (Fisher Scientific) by removing the magnetic field. All steps are performed following the manufacturer's protocol. After completion of both automatic and manual procedures, positively selected cells are counted via flow cytometry and appropriately diluted for plate culture.

UCB-Derived CD34⁺ HSCs Expansion

To achieve expansion, CD34⁺ HSCs isolated from UCB are seeded at different densities (cells/ml) into 6-wells tissue culture plates (Corning, Amsterdam, NL) in a volume of 2 ml/well and are cultured for 12-15 days in Glycostem Basal Growth Medium (GBGM, FertiPro N.V., Veernem, BE) supplemented with 10% human serum (HS, Sanquin), 25 ng/ml recombinant human stem cell factor (rh SCF), FMS-like tyrosine kinase 3 ligand (rh FLT-3L), thrombopoietin (rh TPO) and interleukin-7 (rh IL-7) (all from CellGenix, Freiburg, DE); after day 9, TPO is replaced with 20 ng/ml interleukin-15 (rh IL-15) (CellGenix). Low molecular weight heparin (LMWH) (Clivarin®, Abbott, Wiesbaden, DE) in a final concentration of 25 μg/ml and a low-dose cytokine cocktail consisting of 10 pg/ml granulocyte-macrophage colony-stimulating factor (rh GM-CSF) (CellGenix), 250 pg/ml granulocyte-colony stimulating factor (rh G-CSF)

(Neupogen®, Amgen Europe B.V., Breda, NL) and 50 pg/ml interleukin-6 (rh IL-6) (CellGenix) are added to complete the ‘expansion medium’ cocktail. UCB-CD34⁺ progenitor cultures are refreshed with new medium every 2-3 days and maintained at 37° C., 95% humidity, 5% CO₂ in a cell incubator.

Expanded UCB-Derived CD34⁺ HSCs Differentiation into UCB-Dffector Cells

At day 12-15 of culture, cell differentiation (and further expansion) is induced by switching the culture medium cocktail to ‘differentiation medium’, i.e. GBGM supplemented with 10% HS, 20 ng/ml of IL-7, SCF, IL-15 (CellGenix) and 1000 U/ml interleukin-2 (rh IL-2) (Proleukin®; Chiron, Munchen, DE) and the same low-dose cytokine cocktail described for the expansion medium. UCB-effector cells are cultured at the density of 1.5*10⁶ cells/ml in a volume of 2-5 ml/well and cultures are refreshed with new medium twice per week from day 15 to the endpoint of the culture (day 35/42).

Flow Cytometry

Flow cytometry analysis is performed on a CytoFlex LX (Beckman Coulter Life Sciences, Woerden, NL). This technique is used to determine effector cells and target cells viability, phenotype (via cell-surface markers expression) and to quantitatively determine cell numbers. Cells are stained with antigen-specific fluorochrome-tagged antibodies for 15 minutes at 4° C., then washed and resuspended in FACS buffer composed of 1x PBS (Lonza) supplemented with 0.5% Albuman (Sanquin) and 2 mM EDTA pH 8.0 (Fisher Scientific). To analyse cell viability, the 7-aminoactinomycin D (7-AAD) DNA intercalating marker is used: viable cells, which membrane is not permeable to the dye, are negative, while dying cells, where the dye is able to intercalate with DNA, are positive. Cell populations of interest are initially identified by plotting the forward scatter (FSC) against the side scatter (SSC). Populations of interest are identified on the FSC/SSC plot, then gated for viability (7-AAD⁻) and for positivity for the surface antigen of interest. The antibodies used are: anti-CD45-KromeOrange clone J.33, anti-CD34-PC7 clone 581 (both from Beckman Coulter), anti-CD56-APC-Vio770 clone REA196 (Miltenyi Biotec) and 7-AAD dye (Sigma Aldrich, Zwijndrecht, NL).

Flow Cytometry-Based Effector Cell Potency Assay

Effector cells potency is analysed by co-culturing overnight effector cells and target tumour cell lines and subsequently determining target cell killing via 7-AAD staining and flow cytometry. Target cells are resuspended at 1*10⁷ cells/ml in PBS and pre-labelled with 0.012 mg/ml Pacific Blue™ succinimidyl ester (PBSE, Thermo Fisher Scientific) for 10 minutes in a cell incubator; labelling is stopped by adding 1 volume of target cells culture medium supplemented with 10% FBS. Cells are then washed twice with PBS, counted via flow cytometry and finally resuspended at 1*10⁶ cells/ml in culture medium with 10% FBS. Effector cells are stained with cell-specific surface markers and counted via flow cytometry, then finally resuspended at 1*10⁶ cells/ml in GBGM supplemented with 2% HS. Pre-stained target cells are then co-cultured with effector cells in 96-well tissue culture plates at 1:1 ratio, 5*10⁴ cells/well for each cell type, in a volume of 100 μl, in technical triplicates. Effector cells alone and target cells alone are included as controls. After overnight co-culture in a cell incubator (37° C., 95% humidity, 5% CO₂), samples are diluted 1:1 with FACS buffer, stained with 7-AAD and immediately analysed via flow cytometry. Target cells are distinguished from effector cells through FSC/SSC properties and PBSE positivity; PBSE positive, 7-AAD negative or positive cells (PBSE17-AAD⁻, i.e. viable target cells or PBSE17-AAD+, i.e. dead target cells) are then counted. The average of PBSE/7-AAD⁻ cells from the co-culture triplicates is normalised on the same value obtained for target cells cultured alone to exclude the influence of physiological death of target cells, then the percentage of killed cells is calculated by subtracting the obtained value to 1 and multiplying it by 100.

Statistical Analysis

All statistical analysis is performed using the GraphPad Prism 8 software (Sand Diego, CA). Data is represented as average of independent biological donors (N=4-5), error is calculated as standard deviation (SD). Student's t-test or one-way ANOVA with multiple comparisons correction (Tukey's method) are performed and significance of results is showed as p-values (ns, statistically non-significant; * p≤0.033; ** p≤0.002; *** p≤0.001).

Results

Part 1—Effect of CD34⁺ haematopoietic stem cell (HSC) progenitors' seeding density on in vitro cell expansion in complete culture medium

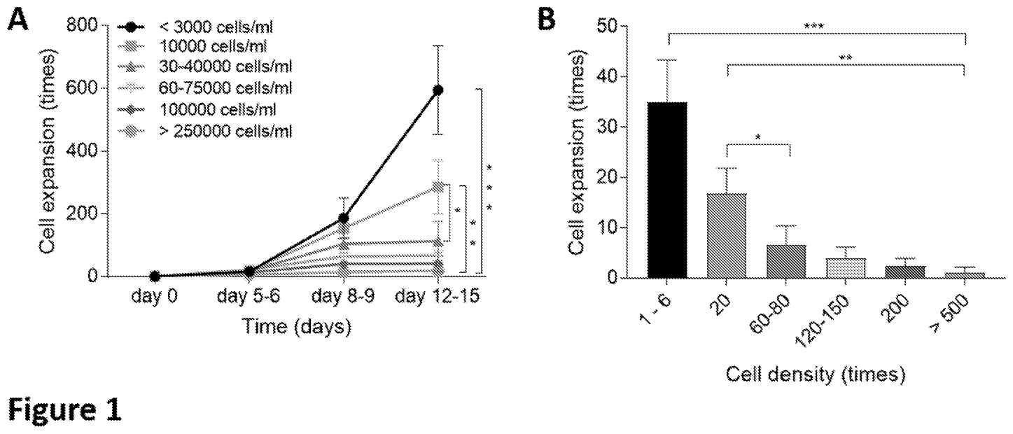

Day 0 human CD34⁺ HSCs isolated from fresh UCB are counted via flow cytometry and diluted at 6 or more different, increasing densities (expressed in cells/ml) for expansion culture in complete medium (Table 1). Cultures are monitored and expansion medium is refreshed regularly until day 12-15; intermediate and final cell expansion is analysed via flow cytometry at days 5-6, 8-9 and 12-15. For every time point, expansion is calculated on day 0 initial cell density for every condition and plotted against time (FIG. 1A). As shown, intermediate and endpoint expansion is higher for lower cell densities, significantly increasing from days 8-9 until the end of the expansion phase. Endpoint expansion ranges from 590 times for less than 3000 CD34⁺ cells/ml to 19 times for more than 250000 CD34⁺ cells/ml. The inverse correlation between cell culture density and expansion is displayed in FIG. 1B, where relative cell expansion is plotted against the progressive relative increase in density. These data show how higher CD34⁺ HSCs expansion is achieved in complete culture medium when cell culture is started at a low density and how progressively reducing the density affects expansion.

Part 2—Effect of CD34⁺ haematopoietic stem cell (HSC) progenitors' expansion density on effector cells differentiation and potency in complete culture medium

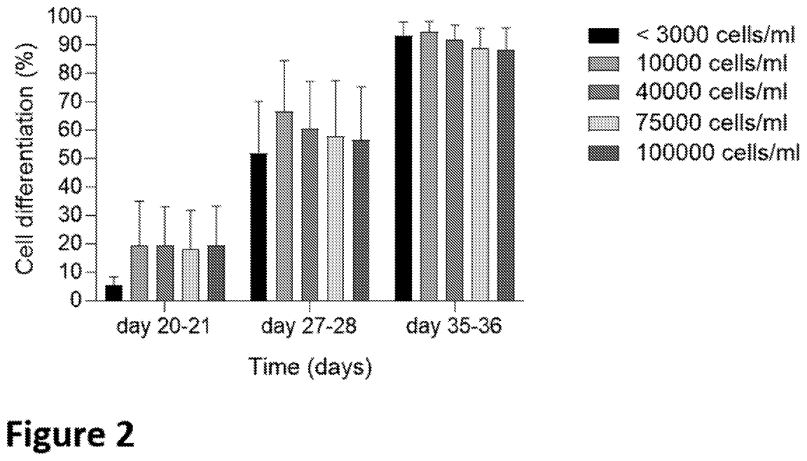

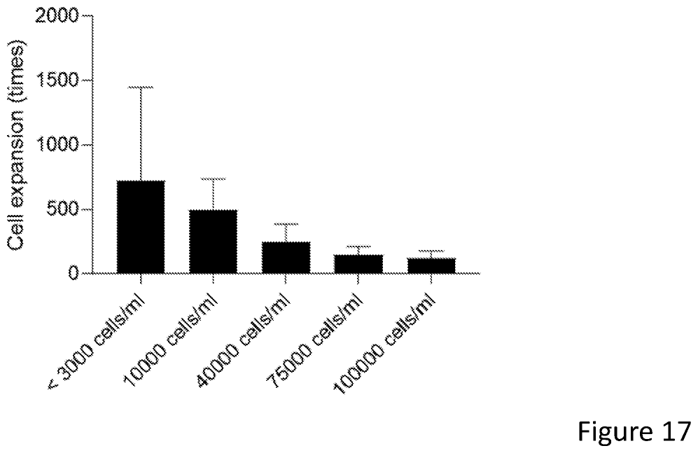

Progressing to differentiation phase, day 12-15 cells from every expansion condition are set at the density of 1.5*10⁶ cells/ml in differentiation medium; medium is refreshed twice per week and effector cells further expansion and phenotype is monitored once every week until day 35-36. This setting allows to examine the influence of the initial expansion on cell differentiation excluding any impact of unequal cell handling during the differentiation phase. FIG. 2 shows how differentiation of effector cells progressively increases for all conditions between days 20 and 35 (from 5-13% to 75-95%). Although the lowest initial density cultures (<3000 CD34⁺ cells/ml) start to differentiate slightly later than higher densities, such gap is largely closed at day 27-28. At day 35, all cells are efficiently differentiated, although the lowest percentage is reached by the 100000 CD34⁺ cells/ml (average 88%) and the highest is reached by 10000 CD34⁺ cells/ml or lower (average of 94%). During differentiation phase, cells are still expanding, although at a slower pace; expansion is maintained higher for the low-density conditions (FIG. 17). These results show how initial low cell density during the expansion phase not only increases cell expansion, but it is also beneficial for the endpoint of differentiation, resulting in higher maturation of effector cells in complete culture medium.

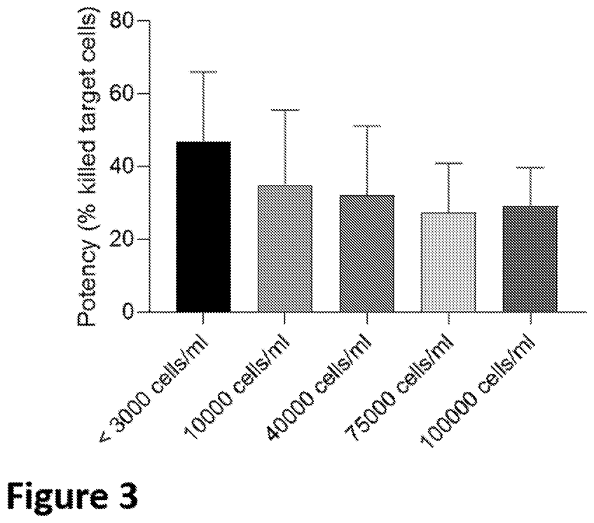

Effector cells potency in the killing of target tumour cells is determined at day 35. All effector cells conditions are co-cultured with tumour cell lines overnight and target cells killing is subsequently determined via flow cytometry. As shown in FIG. 3, 15-72% of target cells are killed by effector cells after overnight co-culture, demonstrating the great potential of effector cells to effectively recognize and eliminate tumour cells. However, low density expansion conditions (in particular <3000 CD34⁺ cells/ml) are more efficient in killing compared to higher densities, showing up to 1.5 fold more potency (<3000 CD34⁺ cells/ml compared with 75000 CD34⁺ cells/ml). Such data indicate that lower expansion density not only improves cell expansion and differentiation of effector cells cultured in complete medium, but also their functionality.

In this first Example, the data obtained from manual and from automatic, closed system selection were pooled, in the next Example, manual and automatic selection are compared to each other.

TABLE 1
Day 0 seeding conditions of CD34⁺ haematopoietic stem cells (HSCs)
purified from fresh umbilical cord blood (UCB). Freshly isolated HSCs
are counted via flow cytometry and diluted at different, increasing cell
concentrations (expressed in cells/ml) in 6-wells tissue culture plates
(in duplicates or triplicates) in a volume of 2 ml/well. Since not all
conditions have been used in all the experiments, similar cell densities
have been pooled as shown in column “Group”. The column
“Concentration” shows the relative cell density between all conditions,
where the lowest, 500 CD34⁺ cells/ml, is set as 1.

| | | | | day 0 |
|---|---|---|---|---|
| CD34⁺ HSC density | Concentration | | | volume |
| (cells/ml) | (times) | Group | | (ml) |
| 500 CD34⁺ cells/ml | 1 | <3000 | cells/ml | 2 |
| 2000 CD34⁺ cells/ml | 4 | | | 2 |
| 3000 cells/ml | 6 | | | 2 |
| 10000 CD34⁺ cells/ml | 20 | 10000 | cells/ml | 2 |
| 30000 CD34⁺ cells/ml | 60 | 30-40000 | cells/ml | 2 |
| 40000 CD34⁺ cells/ml | 80 | | | 2 |
| 60000 CD34⁺ cells/ml | 120 | 60-75000 | cells/ml | 2 |
| 75000 CD34⁺ cells/ml | 150 | | | 2 |
| 100000 CD34⁺ cells/ml | 200 | 100000 | cells/ml | 2 |
| >250000 CD34⁺ cells/ml | >500 | >250000 | cells/ml | 2 |

### Example 2

Introduction

Human CD34⁺ haematopoietic stem cells (HSCs) isolated from fresh umbilical cord blood (UCB) can give rise to effector cells able to kill target tumour cells via a multi-step in vitro process based on an initial expansion phase (days 0-14), followed by an effector cell-specific differentiation phase (days 15-35/42). Optimization of the progenitor's isolation process as well as of expansion and differentiation conditions is useful to generate large batches of highly differentiated effector cells. Here, the influence of the day 0 CD34⁺ HSCs cells isolation method and culture density on the expansion, differentiation and potency of effector cells to kill target cells is analysed. Two methods for CD34⁺ HSCs isolation from blood, one automated, closed method and one manual, open system are compared. Selected progenitors are seeded for expansion phase at 6 different, increasing densities (expressed in cells/ml) for both methods and monitored between days 0 and 15, when final cell expansion is determined via flow cytometry. At day 15, all conditions are set at the same density for the differentiation phase, to determine the effect of the initial expansion density on the subsequent differentiation. At days 20, 28 and 35, effector cells differentiation is monitored via flow cytometry. At day 35, endpoint effector cells' potency is analysed via an in vitro assay where effector cells are co-cultured overnight with target tumour cells and killing of targets is assessed via flow cytometry.

Materials and Methods

Tumour Cell Lines

Tumour cell lines, used in the effector cell potency assay, are cultured in IMDM medium (Iscove's modified Dulbecco's medium, Lonza, Maastricht, NL) containing 100 U/ml penicillin and 100 U/ml streptomycin (Lonza), 2 mM L-Glutamine (Lonza) and 10% foetal bovine serum (FBS, Fisher Scientific, Landsmeer, NL). Cell cultures are passaged every 5 days and maintained at 37° C., 95% humidity, 5% CO₂ in a cell incubator.

CD34⁺ Haematopoietic Stem Cells (HSCs) Isolation from Umbilical Cord Blood (UCB)

A fresh umbilical cord blood unit (supplied by Anthony Nolan, London UK) is divided in two equal volumes and destined for the simultaneous, parallel isolation of CD34⁺ HSCs with two methods, an automatic closed system and a manual open system. Fully automatic selection is performed by using the closed immunomagnetic CliniMACS Prodigy® LP-34 System (Miltenyi Biotec GmbH, Bergisch Gladbach, Germany). The CliniMACS Prodigy TS310 tubing set is installed on the instrument and the CliniMACS PBS/EDTA (Phosphate Buffer Saline/Ethylenediaminetetraacetic acid) buffers are connected. After priming of the instrument, the fresh umbilical cord blood is connected and the CliniMACS CD34 Reagents for labelling of CD34⁺ cells are added. The magnetically labelled CD34⁺ cells are retained in the separation column (in which strong magnetic fields are generated) for washing steps, then eluted in 80 ml of cell culture medium after removing the magnetic field. All steps are performed according to the CliniMACS Prodigy® LP-34 System user manual.

Manual selection is performed by pipetting fresh blood diluted 1:3 with PBS on top of a Ficoll-Paque (GE Healthcare, Hoevelaken, NL) layer in a sterile tube, then centrifuging at 900 g for 30 min at 20° C., with brake turned off. The separated mononuclear cell layer is transferred to a sterile tube using a pipette; cells are then washed twice with PBS (Lonza). Cells are counted via flow cytometry and CD34⁺ cells are labelled with the CD34 MicroBead Kit (Miltenyi Biotec); after labelling, the cells are loaded on a LS Column placed on a MultiStand (both from Miltenyi Biotec) to apply a magnetic field, unlabelled cells are washed, then the CD34⁺ HSCs are eluted in 15 ml of FACS buffer composed of 1x PBS supplemented with 0.5% Albuman (Sanquin, Amsterdam, NL) and 2 mM EDTA pH 8.0 (Thermo Fisher Scientific, Waltham, Mass.) by removing the magnetic field. All steps are performed following the manufacturer's protocol. After completion of both automatic and manual procedures, positively selected cells are counted via flow cytometry and appropriately diluted for plate culture.

UCB-Derived CD34⁺ HSCs Expansion

To achieve expansion, CD34⁺ HSCs isolated from UCB are seeded at different cell densities (expressed in cells/ml) into 6-wells tissue culture plates (Corning, Amsterdam, NL) in a volume of 2 ml/well and are cultured for 15 days in Glycostem Basal Growth Medium (GBGM, FertiPro N.V., Veernem, BE) supplemented with 10% human serum (HS, Sanquin, Amsterdam, NL), 25 ng/ml recombinant human stem cell factor (rh SCF), FMS-like tyrosine kinase 3 ligand (rh Flt-3L), thrombopoietin (rh TPO) and interleukin-7 (rh IL-7) (all from CellGenix, Freiburg, DE); after day 9, TPO is replaced with 20 ng/ml interleukin-15 (rh IL-15) (CellGenix). A low-dose cytokine cocktail consisting of 10 pg/ml granulocyte-macrophage colony-stimulating factor (rh GM-CSF) (CellGenix), 250 pg/ml granulocyte-colony stimulating factor (rh G-CSF) (Neupogen®, Amgen Europe B.V., Breda, NL) and 50 pg/ml interleukin-6 (rh IL-6) (CellGenix) is added to complete the ‘expansion medium’ cocktail. UCB-CD34⁺ progenitor cultures are refreshed with new medium every 2-3 days and maintained at 37° C., 95% humidity, 5% CO₂ in a cell incubator.

Expanded UCB-Derived CD34⁺ HSCs Differentiation into UCB-Effector Cells

At day 14-15 of culture, cell differentiation (and further expansion) is induced by switching the culture medium cocktail to ‘differentiation medium’, i.e. GBGM supplemented with 10% HS, 20 ng/ml of IL-7, SCF, IL-15 (CellGenix) and 1000 U/ml interleukin-2 (rh IL-2) (Proleukin®; Chiron, Munchen, DE) and the same low-dose cytokine cocktail described for the expansion medium. UCB-effector cells are cultured at the density of 1.5*10⁶ cells/ml in a volume of 2-5 ml/well and cultures are refreshed with new medium twice per week from day 15 to the endpoint of the culture (days 35-42).

Flow Cytometry

Flow cytometry analysis is performed on a CytoFlex LX (Beckman Coulter Life Sciences, Woerden, NL). This technique is used to determine effector cells and target cells viability, phenotype (via cell-surface markers expression) and to quantitatively determine cell numbers. Cells are stained with antigen-specific fluorochrome-tagged antibodies for 15 minutes at 4° C., then washed and resuspended in FACS buffer composed of 1x PBS (Lonza) supplemented with 0.5% Albuman (Sanquin) and 2 mM EDTA pH 8.0 (Fisher Scientific). To analyse cell viability, the 7-aminoactinomycin D (7-AAD) DNA intercalating marker is used: viable cells, which membrane is not permeable to the dye, are negative, while dying cells, where the dye is able to intercalate with DNA, are positive. Cell populations of interest are initially identified by plotting the forward scatter (FSC) against the side scatter (SSC). Populations of interest are identified on the FSC/SSC plot, then gated for viability (7-AAD⁻) and for positivity for the surface antigen of interest. The antibodies used are: anti-CD45-KromeOrange clone J.33, anti-CD34-PC7 clone 581 (both from Beckman Coulter), anti-CD56-APC-Vio770 clone REA196 (Miltenyi Biotec) and 7-AAD dye (Sigma Aldrich, Zwijndrecht, NL).

Flow Cytometry-Based Effector Cell Potency Essay

Effector cells potency is analysed by co-culturing effector cells and target tumour cell lines overnight and subsequently determining target cell killing via 7-AAD staining and flow cytometry. Target cells are resuspended at 1*10⁷ cells/ml in PBS and pre-labelled with 0.012 mg/ml Pacific Blue™ succinimidyl ester (PBSE, Thermo Fisher Scientific) for 10 minutes in a cell incubator; labelling is stopped by adding 1 volume of target cells culture medium supplemented with 10% FBS. Cells are then washed twice with PBS, counted via flow cytometry and finally resuspended at 1*10⁶ cells/ml in culture medium with 10% FBS. Effector cells are stained with cell-specific surface markers and counted via flow cytometry, then finally resuspended at 1*10⁶ cells/ml in GBGM supplemented with 2% HS. Pre-stained target cells are then co-cultured with effector cells in 96-well tissue culture plates at 1:1 ratio, 5*10⁴ cells/well for each cell type, in a volume of 100 μl, in technical triplicates. Effector cells alone and target cells alone are included as controls. After overnight co-culture in a cell incubator (37° C., 95% humidity, 5% CO₂), samples are diluted 1:1 with FACS buffer, stained with 7-AAD and immediately analysed via flow cytometry. Target cells are distinguished from effector cells through FSC/SSC properties and PBSE positivity; PBSE positive, 7-AAD negative or positive cells (PBSE17-AAD⁻, i.e. viable target cells or PBSE17-AAD+, i.e. dead target cells) are then counted. The average of PBSE/7-AAD⁻ cells from the co-culture triplicates is normalised on the same value obtained for target cells cultured alone to exclude the influence of physiological death of target cells, then the percentage of killed cells is calculated by subtracting the obtained value to 1 and multiplying it by 100.

Results

Part 1—Effect of CD34⁺ haematopoietic stem cell (HSC) progenitors' isolation method and seeding density on in vitro cell expansion in complete culture medium

One unit of fresh human umbilical cord blood is divided in two equal volumes and CD34⁺ HSCs are isolated in parallel with the CliniMACS Prodigy, an automated closed system (further named: Prodigy) or performing a Ficoll-Paque gradient followed by column separation, a manual, open system (further named: manual). Isolated cells are then counted via flow cytometry and diluted in complete expansion medium for cell culture (see below). Notably, CD34⁺ cells recovery is comparable between the two methods, with 1.08*10⁶ cells harvested from the Prodigy and 0.98*10⁶ cells harvested from the manual procedure. The influence of CD34⁺ isolation method on cell morphology, expansion, differentiation and potency is then investigated.

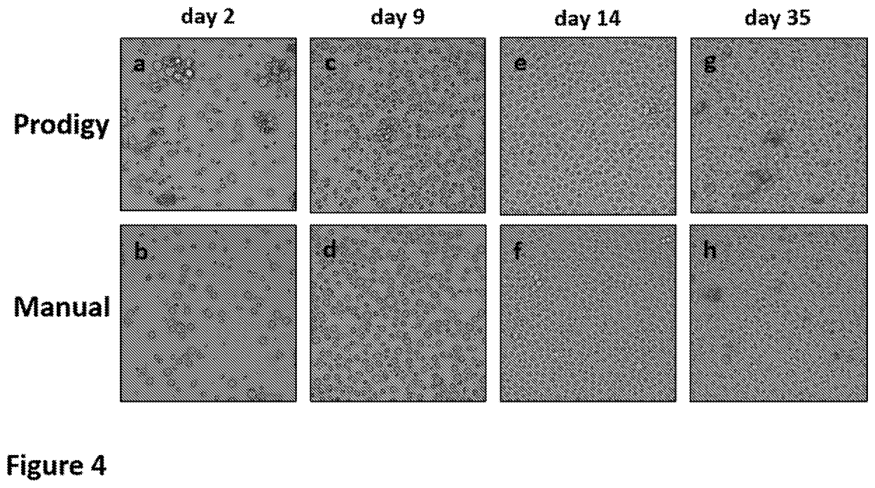

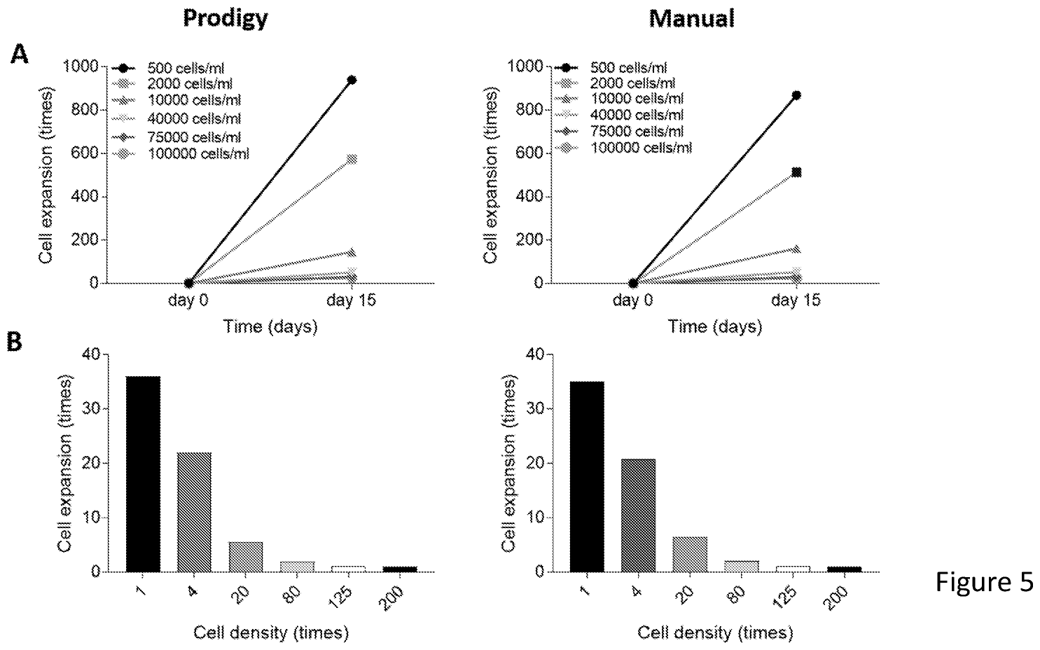

Prodigy- and manually selected CD34⁺ HSCs are then diluted at 6 different, increasing densities (expressed in cells/ml) for expansion culture (Table 2). Visual examination of cells at different time points does not show any difference between the two methods; some red blood cells are present, especially in Prodigy-selected cells, but they disappear after few days of culture and do not impact on cell health, proliferation or differentiation (FIG. 4). During expansion culture, medium is refreshed regularly until day 15, when cell expansion is analysed via flow cytometry. Expansion at day 15 is calculated on day 0 initial cell density for every condition and plotted against time (FIG. 5A). As shown, Prodigy-and manually-selected cells show very similar expansion profiles for all conditions, ranging from very high expansion for lower densities (940 times for Prodigy and 870 for manual for 500 CD34⁺ cells/ml), progressively decreasing to 25 times for 100000 CD34⁺ cells/ml. The inverse correlation trend between cell culture density and expansion is displayed in FIG. 5B, where relative cell expansion is plotted against the progressive relative increase in density for both Prodigy and manual methods. These data show how higher CD34⁺ HSCs expansion is achieved when cell culture is started at a low density, irrespectively of the chosen cell isolation method, and how progressively reducing the density affects expansion.

Part 2—Effect of CD34⁺ haematopoietic stem cell (HSC) progenitors' isolation method and expansion density on effector cells differentiation and potency in complete culture medium

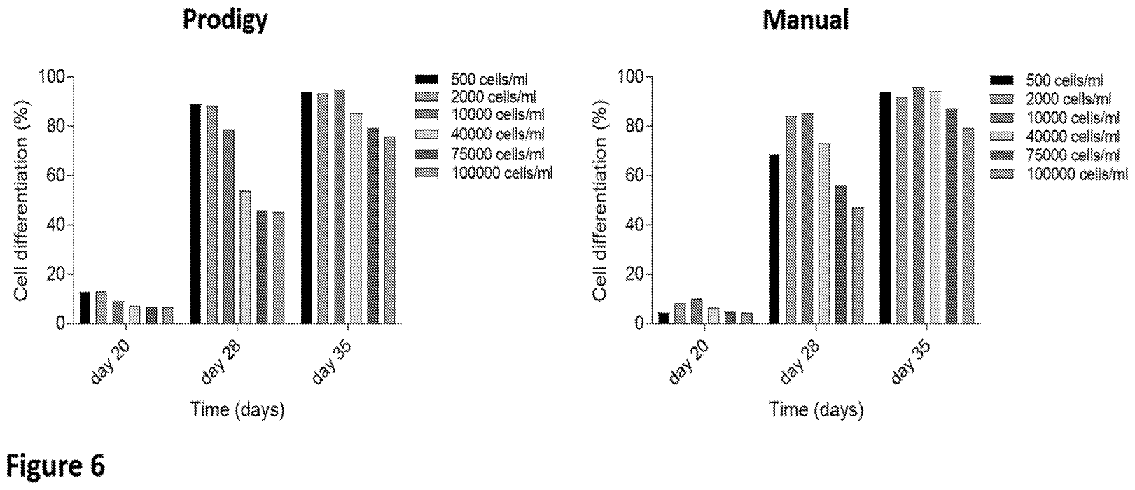

Progressing to differentiation phase, day 15 cells from every expansion condition are set at the density of 1.5*10⁶cells/ml in complete differentiation medium; medium is refreshed twice per week and effector cells further expansion and phenotype is monitored once every week until day 35. This setting allows to examine the influence of the initial expansion on cell differentiation excluding any impact of unequal cell handling during the differentiation phase. FIG. 6 shows how differentiation of effector cells progressively increases for all conditions between days 20 and 35 (from 7-13% for Prodigy and 4-10% for manual at day 20 to 76-95% for Prodigy and 79-96% for manual at day 35). Notably, lower density expansion conditions (500 CD34⁺ cells/ml, 2000 CD34⁺ cells/ml, 10000 CD34⁺ cells/ml) are progressing faster through early differentiation (days 20-28) compared to higher densities (40,000 CD34⁺ cells/ml, 75,000 CD34⁺ cells/ml and 100,000 CD34⁺ cells/ml) in particular for Prodigy-isolated CD34⁺ cells. This gap is largely reduced at day 35, most likely because a plateau of almost 100% differentiation is reached. Nevertheless, lower cell initial cell densities (in particular 500, 2000 and 10,000 CD34⁺ cells/ml seem superior over 40,000 or more (Prodigy) or 75,000 or more (Manual). During differentiation phase, cells are still expanding, although at a slower pace. For both methods, however, expansion is still higher for the low-density conditions (not shown). These results show how initial low cell density during the expansion phase not only increases cell expansion, but also enhances cell differentiation, resulting in faster maturation of effector cells. Notably, the CD34⁺ cells isolation method of choice does not influence this behaviour, since automatic and manual techniques give comparable results.

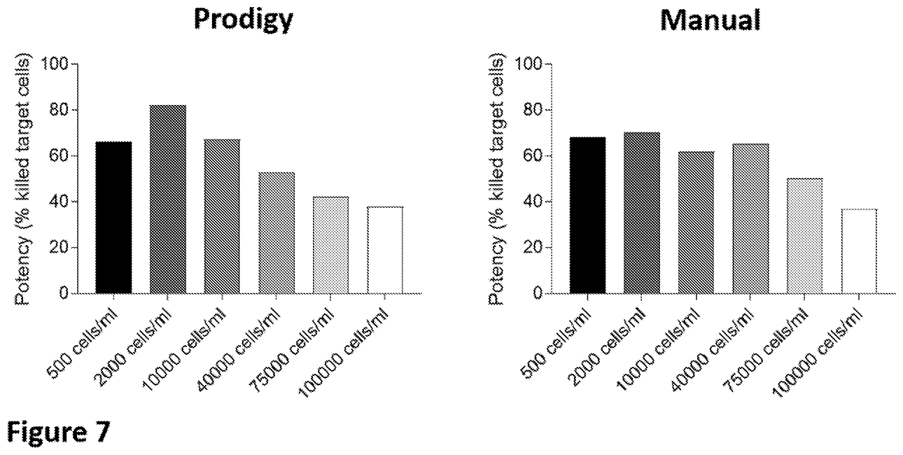

Effector cells potency in killing target tumour cells is determined at day 35. All effector cells conditions are co-cultured overnight with tumour cell lines and target cells killing is subsequently determined via flow cytometry. As shown in FIG. 7, 38-82% for Prodigy and 37-70% for manual of target cells are killed by effector cells after overnight co-culture, demonstrating the great potential of effector cells to effectively recognise and eliminate tumour cells. However, low density expansion conditions (500 CD34⁺ cells/ml, 2000 CD34⁺ cells/ml and 10000 CD34⁺ cells/ml) are more efficient in killing compared to higher densities (40000 CD34⁺ cells/ml, 75000 CD34⁺ cells/ml and 100000 CD34⁺ cells/ml), showing over 60% potency for both methods, but in particular for Prodigy-isolated cells. Such data indicate that lower expansion density not only improves cell expansion and differentiation, but also potency of killing target tumour cells and that the cell isolation method does not have any impact on cell functionality.

TABLE 2
Day 0 seeding conditions of CD34⁺ haematopoietic stem cells (HSCs)
purified from fresh umbilical cord blood (UCB). Freshly Prodigy-or
manually-isolated HSCs are counted via flow cytometry and diluted
at 6 different, increasing cell concentrations (expressed in cells/ml)
in 6-wells tissue culture plates (as single replicates) in a volume of
2 ml/well. The column “Concentration” shows the relative cell density
between all conditions, where the lowest, 500 CD34⁺ cells/ml, is set as 1.

| | | day 0 |
|---|---|---|
| CD34⁺ HSC density | Concentration | volume |
| (cells/ml) | (times) | (ml) |
| 500 CD34⁺ cells/ml | 1 | 2 |
| 2000 CD34⁺ cells/ml | 4 | 2 |
| 10000 CD34⁺ cells/ml | 20 | 2 |
| 40000 CD34⁺ cells/ml | 80 | 2 |
| 75000 CD34⁺ cells/ml | 125 | 2 |
| 100000 CD34⁺ cells/ml | 200 | 2 |

### Example 3

Introduction

Human CD34⁺ haematopoietic stem cells (HSCs) isolated from fresh umbilical cord blood (UCB) can give rise to effector cells able to kill target tumour cells via a multi-step in vitro process based on an initial expansion phase (days 0-14), followed by an effector cell-specific differentiation phase (days 15-35/42). As shown in Examples 1 and 2, cell seeding density at time 0 has a major impact on the expansion, differentiation and potency of effector cells. Low density-seeded cells (below 10000 CD34⁺ cells/ml) have a strong advantage in expansion, differentiation into effector cells and in killing potential against target tumour cells. In this example, the influence of the culture medium composition on such features of effector cells is investigated. During expansion and differentiation phases, cells are cultured without low density cytokine (LDC) cocktail, present in the complete expansion and differentiation medium and consisting of 10 pg/ml granulocyte-macrophage colony-stimulating factor (rh GM-CSF), 250 pg/ml granulocyte-colony stimulating factor (rh G-CSF) and 50 pg/ml interleukin-6 (rh IL-6). Day 0 HSCs are seeded in LDC-free medium at 6 different densities, ranging from 2000 to 100000 CD34⁺ cells/ml, then expansion of progenitors, differentiation and potency of effector cells in such potentially sub-optimal conditions is determined.

Materials and Methods

Tumour Cell Lines

Tumour cell lines, used in the effector cell potency assay, are cultured in IMDM medium (Iscove's modified Dulbecco's medium, Lonza, Maastricht, NL) containing 100 U/ml penicillin and 100 U/ml streptomycin (Lonza), 2 mM L-Glutamine (Lonza) and 10% foetal bovine serum (FBS, Fisher Scientific,

Landsmeer, NL). Cell cultures are passaged every 5 days and maintained at 37° C., 95% humidity, 5% CO₂ in a cell incubator.

CD34⁺ Haematopoietic Stem Cells (HSCs) Isolation from Umbilical Cord Blood (UCB)

CD34⁺ HSCs are isolated from fresh umbilical cord blood units (supplied by Anthony Nolan, London UK) using manual selection. First, fresh blood is diluted 1:3 with PBS and is pipetted on top of a Ficoll-Paque (GE Healthcare, Hoevelaken, NL) layer in a sterile tube for centrifugation at 900 g for 30 min at 20° C., with brake turned off. The separated mononuclear cell layer is transferred to a sterile tube using a pipette; cells are then washed twice with PBS (Lonza). Cells are counted via flow cytometry and CD34⁺ cells are labelled with the CD34 MicroBead Kit (Miltenyi Biotec); after labelling, the cells are loaded on a LS Column placed on a MultiStand (both from Miltenyi Biotec) to apply a magnetic field, unlabelled cells are washed, then the CD34⁺ HSCs are eluted in 15 ml of FACS buffer composed of 1x PBS supplemented with 0.5% Albuman (Sanquin, Amsterdam, NL) and 2 mM EDTA pH 8.0 (Thermo Fisher Scientific, Waltham, MA) by removing the magnetic field. All steps are performed following the manufacturer's protocol. After selection, cells are counted via flow cytometry and appropriately diluted for plate culture.

UCB-Derived CD34⁺ HSCs Expansion

To achieve expansion, CD34⁺ HSCs isolated from UCB are seeded at different cell densities (expressed in cells/ml) into 6-wells tissue culture plates (Corning, Amsterdam, NL) in a volume of 2 ml/well and are cultured for 13-14 days in Glycostem Basal Growth Medium (GBGM, FertiPro N.V., Veernem, BE) supplemented with 10% human serum (HS, Sanquin, Amsterdam, NL), 25 ng/ml recombinant human stem cell factor (rh SCF), FMS-like tyrosine kinase 3 ligand (rh Flt-3L), thrombopoietin (rh TPO) and interleukin-7 (rh IL-7) (all from CellGenix, Freiburg, DE) to complete the ‘LDC-free expansion medium’ cocktail; after day 9, TPO is replaced with 20 ng/ml interleukin-15 (rh IL-15) (CellGenix). UCB-CD34⁺ cultures are refreshed with new medium every 2-3 days and maintained at 37° C., 95% humidity, 5% CO₂ in a cell incubator.

Expanded UCB-Derived CD34⁺ HSCs Differentiation into UCB-Effector Cells

At day 13-14 of culture, cell differentiation (and further expansion) is induced by switching the culture medium cocktail to ‘LDC-free differentiation medium’, i.e. GBGM supplemented with 10% HS, 20 ng/ml of IL-7, SCF, IL-15 (CellGenix) and 1000 U/ml interleukin-2 (rh IL-2) (Proleukin®; Chiron, München, DE). UCB-effector cells are cultured at the density of 1.5*10⁶ cells/ml in a volume of 2-5 ml/well and cultures are refreshed with new medium twice per week from day 13-14 to the endpoint of the culture (days 35-42).

Flow Cytometry

Flow cytometry analysis is performed on a CytoFlex LX (Beckman Coulter Life Sciences, Woerden, NL). This technique is used to determine effector cells and target cells viability, phenotype (via cell-surface markers expression) and to quantitatively determine cell numbers. Cells are stained with antigen-specific fluorochrome-tagged antibodies for 15 minutes at 4° C., then washed and resuspended in FACS buffer composed of 1x PBS (Lonza) supplemented with 0.5% Albuman (Sanquin) and 2 mM EDTA pH 8.0 (Fisher Scientific). To analyse cell viability, the 7-aminoactinomycin D (7-AAD) DNA intercalating marker is used: viable cells, which membrane is not permeable to the dye, are negative, while dying cells, where the dye is able to intercalate with DNA, are positive. Cell populations of interest are initially identified by plotting the forward scatter (FSC) against the side scatter (SSC). Populations of interest are identified on the FSC/SSC plot, then gated for viability (7-AAD⁻) and for positivity for the surface antigen of interest. The antibodies used are: anti-CD45-KromeOrange clone J.33, anti-CD34-PC7 clone 581 (both from Beckman Coulter), anti-CD56-APC-Vio770 clone REA196 (Miltenyi Biotec) and 7-AAD dye (Sigma Aldrich, Zwijndrecht, NL).

Flow Cytometry-Based Effector Cell Potency Assay

Effector cells potency is analysed by co-culturing effector cells and target tumour cell lines overnight and subsequently determining target cell killing via 7-AAD staining and flow cytometry. Target cells are resuspended at 1*10⁷ cells/ml in PBS and pre-labelled with 0.012 mg/ml Pacific Blue™ succinimidyl ester (PBSE, Thermo Fisher Scientific) for 10 minutes in a cell incubator; labelling is stopped by adding 1 volume of target cells culture medium supplemented with 10% FBS. Cells are then washed twice with PBS, counted via flow cytometry and finally resuspended at 1*10⁶ cells/ml in culture medium with 10% FBS. Effector cells are stained with cell-specific surface markers and counted via flow cytometry, then finally resuspended at 1*10⁶ cells/ml in GBGM supplemented with 2% HS. Pre-stained target cells are then co-cultured with effector cells in 96-well tissue culture plates at 1:1 ratio, 5*10⁴ cells/well for each cell type, in a volume of 100 in technical triplicates. Effector cells alone and target cells alone are included as controls. After overnight co-culture in a cell incubator (37° C., 95% humidity, 5% CO₂), samples are diluted 1:1 with FACS buffer, stained with 7-AAD and immediately analysed via flow cytometry. Target cells are distinguished from effector cells through FSC/SSC properties and PBSE positivity; PBSE positive, 7-AAD negative or positive cells (PBSE⁺/7-AAD⁻, i.e. viable target cells or PBSE⁺/7-AAD⁺, i.e. dead target cells) are then counted. The average of PBSE⁺/7-AAD⁻ cells from the co-culture triplicates is normalised on the same value obtained for target cells cultured alone to exclude the influence of physiological death of target cells, then the percentage of killed cells is calculated by subtracting the obtained value to 1 and multiplying it by 100.

Statistical Analysis

All statistical analysis is performed using the GraphPad Prism 8 software (Sand Diego, Calif.). Data is represented as average of independent biological donors (N=3-4), error is calculated as standard deviation (SD). Student's t-test or one-way ANOVA with multiple comparisons correction (Tukey's method) are performed and significance of results is showed as p-values (ns, statistically non-significant: p>0.12; * p≤0.033; ** p≤0.002; *** p≤0.001).

Results

Part 1—Effect of CD34⁺ haematopoietic stem cell (HSC) progenitors' seeding density on in vitro cell expansion in LDC-free culture medium

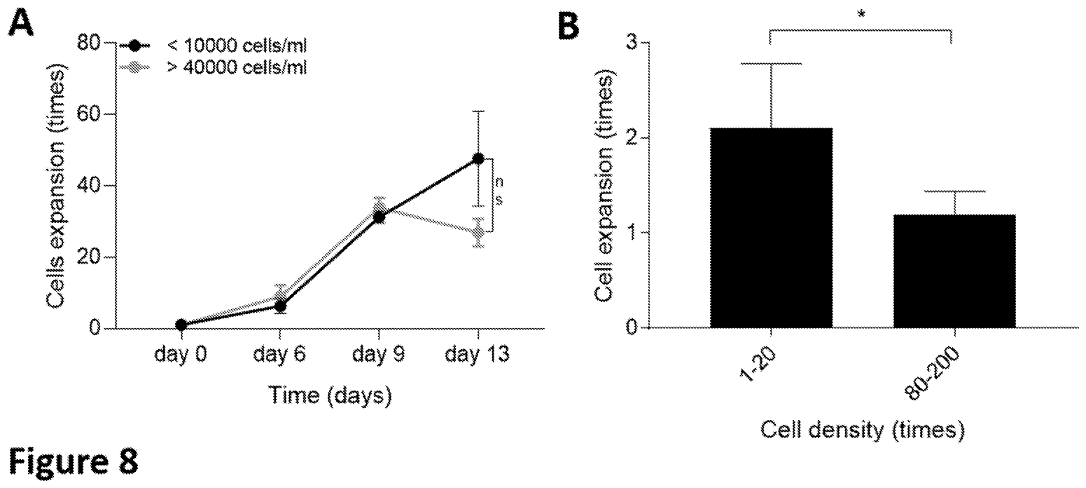

Day 0 human CD34⁺ HSCs isolated from fresh UCB, counted via flow cytometry and diluted at 6 different, increasing densities (expressed as cells/ml), are seeded for expansion culture in LDC-free medium (Table 3). Cultures are monitored and medium is refreshed regularly until day 13-14; intermediate and final cell expansion is analysed via flow cytometry at days 6, 9 and 13-14. For every time point, expansion is calculated on day 0 initial cell density for every condition; low density (500 CD34⁺ cells/ml, 2000 CD34⁺ cells/ml and 10000 CD34⁺ cells/ml, shown as <10000 CD34⁺ cells/ml) and high density settings (40000 CD34⁺ cells/ml, 75000 CD34⁺ cells/ml and 100000 CD34⁺ cells/ml, shown as >40000 CD34⁺ cells/ml) are combined and the average expansion of the two groups is plotted against time (FIG. 8A). As shown, expansion in LDC-free medium is overall lower if compared to complete medium (Examples 1 and 2). However, even uner such sub-optimal conditions, higher expansion is achieved by low density-seeded cells. Endpoint expansion reaches on average 57 times for <10000 CD34⁺ cells/ml or 22 times for >40000 CD34⁺ cells/ml. The inverse correlation between cell culture density and expansion is displayed in FIG. 8B, where relative cell expansion is plotted against the progressive relative increase in density for the two groups. Such results show that, even in LDC-free culture conditions, higher CD34⁺ HSCs expansion is achieved if cells are seeded at a low density.

Part 2—Effect of CD34⁺ haematopoietic stem cell (HSC) progenitors' expansion density on effector cells differentiation and potency in LDC-free culture medium

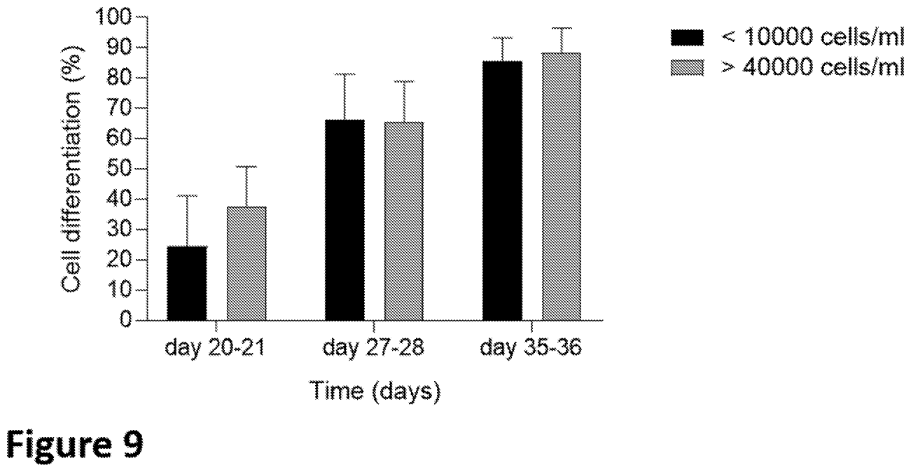

For differentiation phase, day 13-14 cells from every expansion condition are set at the density of 1.5*10⁶ cells/ml in LDC-free differentiation medium; cells obtained from the 500 CD34⁺ cells/mland 2000 CD34⁺ cells/ml initiated cultures are combined due to the low number of cells. As in regular culture, medium refreshment is performed twice and cell count and phenotypic analysis once every week until day 35-36. The ability of the cells to differentiate into mature effector cells and to further expand is monitored. FIG. 9 shows how, overall, cells cultured in LDC-free differentiation medium progressively maturate into effector cells between days 20 and 35 (from 20-38% at day 20-21 to 85-89% at day 35-36). No statistically significant difference in differentiation rate is observed between low density- and high density-cultured cells. Expansion of differentiating cells, however, is maintained slightly higher for low density conditions (not shown).

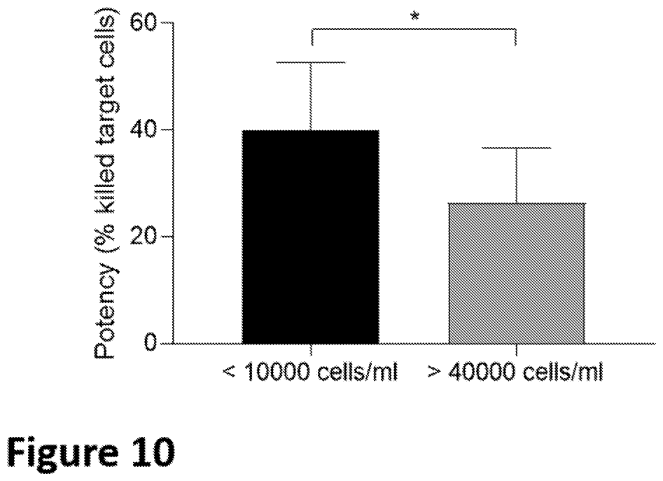

At day 35, effector cells' ability to kill target tumour cells is analysed in a potency assay. All effector cells conditions are co-cultured overnight with tumour cell lines and target cells killing is subsequently determined via flow cytometry. As shown in FIG. 10, 12-62% of target cells are killed by effector cells after overnight co-culture in a 1:1 effector:target ratio, indicating that functional effector cells are generated in any condition. However, low density expansion conditions (<10000 CD34⁺ cells/ml) are significantly more efficient in killing compared to higher densities (>40000 CD34⁺ cells/ml), showing a 1.5-fold higher potency. In conclusion, even though cells are challenged to a greater extent in LDC-free conditions, low density seeding is consistently beneficial to achieving higher numbers of functional effector cells.

TABLE 3
Day 0 seeding conditions of CD34⁺ haematopoietic stem cells (HSCs)
purified from fresh umbilical cord blood (UCB) and cultured in
LDC-free medium. Freshly isolated HSCs are counted via flow
cytometry and diluted at 6 different, increasing cell concentrations
(expressed in cells/ml) in 6-wells tissue culture plates (as single
replicates) in a volume of 2 ml/well. The column “Concentration”
shows the relative cell density between all conditions, where the
lowest, 500 CD34⁺ cells/ml, is set as 1.

| | | | day 0 |
|---|---|---|---|
| CD34⁺ HSC density | Concentration | | volume |
| (cells/ml) | (times) | Group | (ml) |
| 500 CD34⁺ cells/ml | 1 | <10000 cells/ml | 2 |
| 2000 CD34⁺ cells/ml | 4 | | 2 |
| 10000 CD34⁺ cells/ml | 20 | | 2 |
| 40000 CD34⁺ cells/ml | 80 | >40000 cells/ml | 2 |
| 75000 CD34⁺ cells/ml | 125 | | 2 |
| 100000 CD34⁺ cells/ml | 200 | | 2 |

### Example 4

Introduction

Human CD34⁺ haematopoietic stem cells (HSCs) isolated from fresh umbilical cord blood (UCB) can give rise to effector cells able to kill target tumour cells via a multi-step in vitro process based on an initial expansion phase (days 0-14), followed by an effector cell-specific differentiation phase (days 15-35/42). As shown in Examples 1 and 2, day 0 UCB-derived CD34⁺ HSCs culture density has a major impact on the expansion, differentiation and potency of effector cells. Low density-seeded cells (below 10000 CD34⁺ cells/ml) have a strong advantage in expansion, differentiation into effector cells and in killing potential against target tumour cells. Interestingly, as shown in Example 3, sub-optimal culture conditions induced by the removal of the low-density cytokine (LDC) cocktail from the complete medium still support such beneficial effect of low-density culture. In this example, cells are challenged to a greater extent as the serum concentration in the medium is reduced from 10% to 2%. To somehow compensate for serum withdrawal, cytokine concentrations is increased 5 times compared to the complete medium, while LDC is excluded. Day 0 HSCs are seeded in 2% HS, 5x cytokines, LDC-free medium at 2 different densities, low (2000 CD34⁺ cells/ml) or high (40000 CD34⁺ cells/ml), then expansion of progenitors, differentiation and potency of effector cells in such sub-optimal conditions is analysed.

Materials and Methods

Tumour Cell Lines

Tumour cell lines, used in the effector cell potency assay, are cultured in IMDM medium (Iscove's modified Dulbecco's medium, Lonza, Maastricht, NL) containing 100 U/ml penicillin and 100 U/ml streptomycin (Lonza), 2 mM L-Glutamine (Lonza) and 10% foetal bovine serum (FBS, Fisher Scientific, Landsmeer, NL). Cell cultures are passaged every 5 days and maintained at 37° C., 95% humidity, 5% CO₂ in a cell incubator.

CD34⁺ Haematopoietic Stem Cells (HSCs) Isolation from Umbilical Cord Blood (UCB)

CD34⁺ HSCs are isolated from fresh umbilical cord blood units (supplied by Anthony Nolan, London UK) using manual selection. First, fresh blood is diluted 1:3 with PBS and is pipetted on top of a Ficoll-Paque (GE Healthcare, Hoevelaken, NL) layer in a sterile tube for centrifugation at 900 g for 30 min at 20° C., with brake turned off. The separated mononuclear cell layer is transferred to a sterile tube using a pipette; cells are then washed twice with PBS (Lonza). Cells are counted via flow cytometry and CD34⁺ cells are labelled with the CD34 MicroBead Kit (Miltenyi Biotec); after labelling, the cells are loaded on a LS Column placed on a MultiStand (both from Miltenyi Biotec) to apply a magnetic field, unlabelled cells are washed, then the CD34⁺ HSCs are eluted in 15 ml of FACS buffer composed of 1x PBS supplemented with 0.5% Albuman (Sanquin, Amsterdam, NL) and 2 mM EDTA pH 8.0 (Thermo Fisher Scientific, Waltham, Mass.) by removing the magnetic field. All steps are performed following the manufacturer's protocol. After selection, cells are counted via flow cytometry and appropriately diluted for plate culture.

UCB-Derived CD34⁺ HSCs Expansion

To achieve expansion, CD34⁺ HSCs isolated from UCB are seeded at two different cell densities (expressed in cells/ml) into 6-wells tissue culture plates (Corning, Amsterdam, NL) in a volume of 2 ml/well and are cultured for 14 days in Glycostem Basal Growth Medium (GBGM, FertiPro N.V., Veernem, BE) supplemented with 2% human serum (HS, Sanquin, Amsterdam, NL), 125 nem! recombinant human stem cell factor (rh SCF), FMS-like tyrosine kinase 3 ligand (rh Flt-3L), thrombopoietin (rh TPO) and interleukin-7 (rh IL-7) (all from CellGenix, Freiburg, DE) to complete the ‘2% HS, 5x cytokines, LDC-free expansion medium’ cocktail; after day 9, TPO is replaced with 100 nem! interleukin-15 (rh IL-15) (CellGenix). UCB-CD34⁺ cultures are refreshed with new medium every 2-3 days and maintained at 37° C., 95% humidity, 5% CO₂ in a cell incubator. Expanded UCB-derived CD34⁺ HSCs differentiation into UCB-effector cells

At day 14 of culture, cell differentiation (and further expansion) is induced by switching the culture medium cocktail to ‘2% HS, 5x cytokines, LDC-free differentiation medium’, i.e. GBGM supplemented with 2% HS, 100 nem! of IL-7, SCF, IL-15 (CellGenix) and 5000 U/ml interleukin-2 (rh IL-2) (Proleukin®; Chiron, Munchen, DE). UCB-effector cells are cultured at the density of 1.5*10⁶cells/ml in a volume of 2-5 ml/well and cultures are refreshed with new medium twice per week from day 14 to the endpoint of the culture (days 35-42).

Flow Cytometry

Flow cytometry analysis is performed on a CytoFlex LX (Beckman Coulter Life Sciences, Woerden, NL). This technique is used to determine effector cells and target cells viability, phenotype (via cell-surface markers expression) and to quantitatively determine cell numbers. Cells are stained with antigen-specific fluorochrome-tagged antibodies for 15 minutes at 4° C., then washed and resuspended in FACS buffer composed of 1x PBS (Lonza) supplemented with 0.5% Albuman (Sanquin) and 2 mM EDTA pH 8.0 (Fisher Scientific). To analyse cell viability, the 7-aminoactinomycin D (7-AAD) DNA intercalating marker is used: viable cells, which membrane is not permeable to the dye, are negative, while dying cells, where the dye is able to intercalate with DNA, are positive. Cell populations of interest are initially identified by plotting the forward scatter (FSC) against the side scatter (SSC). Populations of interest are identified on the FSC/SSC plot, then gated for viability (7-AAD⁻) and for positivity for the surface antigen of interest. The antibodies used are: anti-CD45-KromeOrange clone J.33, anti-CD34-PC7 clone 581 (both from Beckman Coulter), anti-CD56-APC-Vio770 clone REA196 (Miltenyi Biotec) and 7-AAD dye (Sigma Aldrich, Zwijndrecht, NL).

Flow Cytometry-Based Effector Cell Potency Assay

Effector cells potency is analysed by co-culturing effector cells and target tumour cell lines overnight and subsequently determining target cell killing via 7-AAD staining and flow cytometry. Target cells are resuspended at 1*10⁷ cells/ml in PBS and pre-labelled with 0.012 mg/ml Pacific Blue™ succinimidyl ester (PBSE, Thermo Fisher Scientific) for 10 minutes in a cell incubator; labelling is stopped by adding 1 volume of target cells culture medium supplemented with 10% FBS. Cells are then washed twice with PBS, counted via flow cytometry and finally resuspended at 1*10⁶ cells/ml in culture medium with 10% FBS. Effector cells are stained with cell-specific surface markers and counted via flow cytometry, then finally resuspended at 1*10⁶ cells/ml in GBGM supplemented with 2% HS. Pre-stained target cells are then co-cultured with effector cells in 96-well tissue culture plates at 1:1 ratio, 5*10⁴ cells/well for each cell type, in a volume of 100 μl, in technical triplicates. Effector cells alone and target cells alone are included as controls. After overnight co-culture in a cell incubator (37° C., 95% humidity, 5% CO₂), samples are diluted 1:1 with FACS buffer, stained with 7-AAD and immediately analysed via flow cytometry. Target cells are distinguished from effector cells through FSC/SSC properties and PBSE positivity; PBSE positive, 7-AAD negative or positive cells (PBSE/7-AAD⁻, i.e. viable target cells or PBSE17-AAD+, i.e. dead target cells) are then counted. The average of PBSE/7-AAD⁻ cells from the co-culture triplicates is normalised on the same value obtained for target cells cultured alone to exclude the influence of physiological death of target cells, then the percentage of killed cells is calculated by subtracting the obtained value to 1 and multiplying it by 100.

Statistical Analysis

All statistical analysis is performed using the GraphPad Prism 8 software (Sand Diego, Calif.). Data is represented as average of independent biological donors (N=2), error is calculated as standard deviation (SD). Student's t-test or one-way ANOVA with multiple comparisons correction (Tukey's method) are performed and significance of results is showed as p-values (ns, statistically non-significant: p>0.12; * p≤0.033; ** p≤0.002; *** p≤0.001).

Results

Part 1—Effect of CD34⁺ haematopoietic stem cell (HSC) progenitors' seeding density on in vitro cell expansion in 2% serum, 5x cytokines and LDC-free culture medium

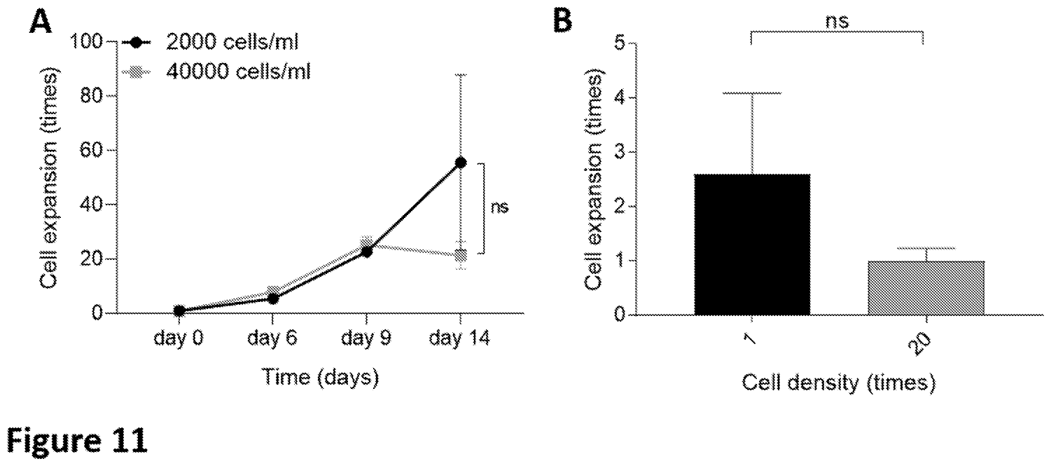

Day 0 human CD34⁺ HSCs isolated from fresh UCB, counted via flow cytometry and diluted at 2 densities, low (2000 CD34⁺ cells/ml) or high (40000 CD34⁺ cells/ml), are seeded for expansion culture in 2% serum, 5x cytokines and LDC-free medium (Table 4). Cultures are monitored and medium is refreshed regularly until day 14; intermediate and final cell expansion is analysed via flow cytometry at days 6, 9 and 14. For every time point, expansion is calculated on day 0 initial cell density and the average expansion of the two conditions is plotted against time (FIG. 11A). Interestingly, cell expansion potential in 2% HS, 5x cytokines, LDC-free medium is comparable to what obtained in LDC-free medium (as shown in Example 3) and, similarly, is increased in the low-density cultures. Endpoint expansion reaches on average 57 times for 2000 CD34⁺cells/mland 22 times for 10000 CD34⁺ cells/ml. Inverse correlation between cell density and relative expansion is showed in FIG. 11B. Overall, even if serum availability is reduced, higher expansion is achieved if cells are seeded at a low density.

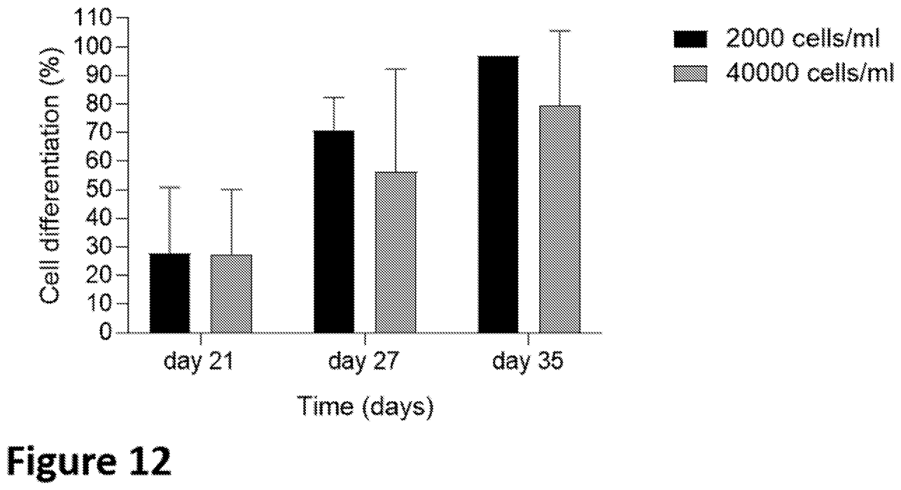

Part 2—Effect of CD34⁺ haematopoietic stem cell (HSC) progenitors' expansion density on effector cells differentiation and potency in 2% serum, 5x cytokines and LDC-free culture medium For differentiation phase, day 14 cells are set at the density of 1.5*10⁶ cells/ml in 2% serum, 5x cytokines and LDC-free differentiation medium. The ability of the cells to differentiate into mature effector cells and to further expand is monitored every week until day 35 and medium is refreshed twice every week. FIG. 12 shows the progression in cell differentiation from day 21 to 35 (from 11-44% at day 21 to 60-96% at day 35). A slight differentiation advantage is observed at later stages for cells expanded as 2000 CD34⁺ cells/ml when compared to 40,000 CD34⁺ cells/ml. Day 35 expansion, similarly, is higher for the lower-density initiated cultures (not shown).

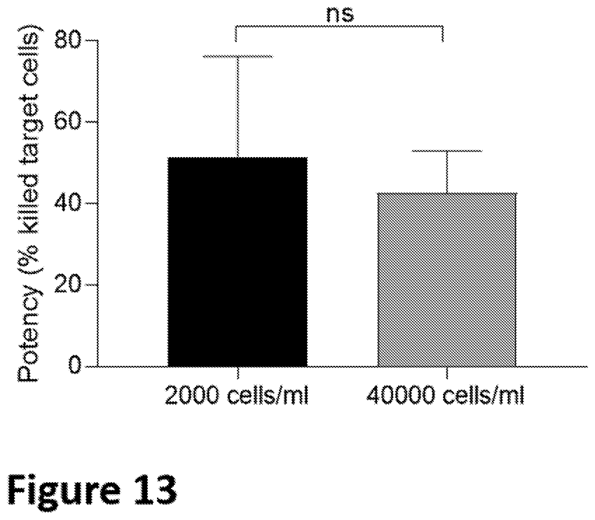

Effector cells potency, tested at day 35, confirms how low-density seeding gives an advantage in the killing of target tumour cells (FIG. 13). On average, 51% of target cells are killed by 2000 CD34⁺ cells/ml effector cells after overnight co-culture in a 1:1 E:T ratio, where 43% are killed by 40000 CD34⁺ cells/ml effector cells (1.2 fold increase in potency). This example strengthens the preference for low seeding density of CD34⁺ HSCs even in conditions in which cells are not able to exploit their full potentials, as serum deprived, high cytokine and LDC-free setting.

This example shows how low seeding density of day 0 CD34⁺ haematopoietic stem cells is beneficial for progenitor cells' expansion, effector cells' maturation and potency even when cells are maintained in sub-optimal conditions, as serum-deprived, high cytokine, LDC-free culture conditions. Such culture conditions are imposing a challenge on cells' ability to expand, differentiate and kill target cells, with cell expansion being the most affected process. Despite the overall drop in cell expansion (up to 10-fold), low density cells maintain an advantage over high density, which is carried throughout differentiation and in functionality.

TABLE 4
Day 0 seeding conditions of CD34⁺ haematopoietic stem cells (HSCs)
purified from fresh umbilical cord blood (UCB) and cultured in 2%
serum, 5× cytokines and LDC-free medium. Freshly isolated HSCs
are counted via flow cytometry and diluted at 2 different, increasing
cell concentrations (expressed in cells/ml) in 6-wells tissue culture
plates (as single replicates) in a volume of 2 ml/well. The column
“Concentration” shows the relative cell density between all conditions,
where the lowest, 2000 CD34⁺ cells/ml, is set as 1.

| | | day 0 |
|---|---|---|
| CD34⁺ HSC density | Concentration | volume |
| (cells/ml) | (times) | (ml) |
| 2000 CD34⁺ cells/ml | 1 | 2 |
| 40000 CD34⁺ cells/ml | 20 | 2 |

### Example 5

Introduction

Human CD34⁺ haematopoietic stem cells (HSCs) isolated from fresh umbilical cord blood (UCB) can give rise to effector cells able to kill target tumour cells via a multi-step in vitro process based on an initial expansion phase (days 0-14), followed by an effector cell-specific differentiation phase (days 15-35/42). As shown in Examples 1 and 2, day 0 UCB-derived CD34⁺ HSCs culture density has a major impact on the expansion, differentiation and potency of effector cells. Low density-seeded cells (below 10000 CD34⁺ cells/ml) have a strong advantage in expansion, differentiation into effector cells and in killing potential against target tumour cells. Interestingly, as shown in Examples 3 and 4, sub-optimal culture conditions induced by the removal of the low-density cytokine (LDC) cocktail from the complete medium or, further, combined with serum withdrawal and increased cytokine concentration, still support such beneficial effect of low-density culture. In this example, the culture conditions are further severed as in the medium, supplemented with 2% human serum, the serum reduction is not compensated by higher cytokine concentrations. LDC mix is excluded as well. As in example 4, day 0 HSCs are seeded in 2% HS, 1x cytokines, LDC-free medium at 2 different densities, low (2000 CD34⁺ cells/ml) or high (40000 CD34⁺ cells/ml), then expansion of progenitors, differentiation and potency of effector cells in such culture conditions is determined.

Materials and Methods

Tumour Cell Lines

Tumour cell lines, used in the effector cell potency assay, are cultured in IMDM medium (Iscove's modified Dulbecco's medium, Lonza, Maastricht, NL) containing 100 U/ml penicillin and 100 U/ml streptomycin (Lonza), 2 mM L-Glutamine (Lonza) and 10% foetal bovine serum (FBS, Fisher Scientific, Landsmeer, NL). Cell cultures are passaged every 5 days and maintained at 37° C., 95% humidity, 5% CO₂ in a cell incubator.

CD34⁺ Haematopoietic Stem Cells (HSCs) Isolation from Umbilical Cord Blood (UCB)

CD34⁺ HSCs are isolated from fresh umbilical cord blood units (supplied by Anthony Nolan, London UK) using manual selection. First, fresh blood is diluted 1:3 with PBS and is pipetted on top of a Ficoll-Paque (GE Healthcare, Hoevelaken, NL) layer in a sterile tube for centrifugation at 900 g for 30 min at 20° C., with brake turned off. The separated mononuclear cell layer is transferred to a sterile tube using a pipette; cells are then washed twice with PBS (Lonza). Cells are counted via flow cytometry and CD34⁺ cells are labelled with the CD34 MicroBead Kit (Miltenyi Biotec); after labelling, the cells are loaded on a LS Column placed on a MultiStand (both from Miltenyi Biotec) to apply a magnetic field, unlabelled cells are washed, then the CD34⁺ HSCs are eluted in 15 ml of FACS buffer composed of 1x PBS supplemented with 0.5% Albuman (Sanquin, Amsterdam, NL) and 2 mM EDTA pH 8.0 (Thermo Fisher Scientific, Waltham, MA) by removing the magnetic field. All steps are performed following the manufacturer's protocol. After selection, cells are counted via flow cytometry and appropriately diluted for plate culture.

UCB-Derived CD34⁺ HSCs Expansion

To achieve expansion, CD34⁺ HSCs isolated from UCB are seeded at two different cell densities (expressed in cells/ml) into 6-wells tissue culture plates (Corning, Amsterdam, NL) in a volume of 2 ml/well and are cultured for 14 days in Glycostem Basal Growth Medium (GBGM, FertiPro N.V., Veernem, BE) supplemented with 2% human serum (HS, Sanquin, Amsterdam, NL), 25 ng/ml recombinant human stem cell factor (rh SCF), FMS-like tyrosine kinase 3 ligand (rh Flt-3L), thrombopoietin (rh TPO) and interleukin-7 (rh IL-7) (all from CellGenix, Freiburg, DE) to complete the ‘2% HS, 1x cytokines, LDC-free expansion medium’ cocktail; after day 9, TPO is replaced with 20 nem! interleukin-15 (rh IL-15) (CellGenix). UCB-CD34⁺ cultures are refreshed with new medium every 2-3 days and maintained at 37° C., 95% humidity, 5% CO₂ in a cell incubator.

Expanded UCB-Derived CD34⁺ HSCs Differentiation into UCB-Effector Cells

At day 14 of culture, cell differentiation (and further expansion) is induced by switching the culture medium cocktail to ‘2% HS, 1x cytokines, LDC-free differentiation medium’, i.e. GBGM supplemented with 2% HS, 20 nem! of IL-7, SCF, IL-15 (CellGenix) and 1000 U/ml interleukin-2 (rh IL-2) (Proleukin®; Chiron, Munchen, DE). UCB-effector cells are cultured at the density of 1.5*10⁶ cells/ml in a volume of 2-5 ml/well and cultures are refreshed with new medium twice per week from day 14 to the endpoint of the culture (days 35-42).

Flow Cytometry

Flow cytometry analysis is performed on a CytoFlex LX (Beckman Coulter Life Sciences, Woerden, NL). This technique is used to determine effector cells and target cells viability, phenotype (via cell-surface markers expression) and to quantitatively determine cell numbers. Cells are stained with antigen-specific fluorochrome-tagged antibodies for 15 minutes at 4° C., then washed and resuspended in FACS buffer composed of 1x PBS (Lonza) supplemented with 0.5% Albuman (Sanquin) and 2 mM EDTA pH 8.0 (Fisher Scientific). To analyse cell viability, the 7-aminoactinomycin D (7-AAD) DNA intercalating marker is used: viable cells, which membrane is not permeable to the dye, are negative, while dying cells, where the dye is able to intercalate with DNA, are positive. Cell populations of interest are initially identified by plotting the forward scatter (FSC) against the side scatter (SSC). Populations of interest are identified on the FSC/SSC plot, then gated for viability (7-AAD⁻) and for positivity for the surface antigen of interest. The antibodies used are: anti-CD45-KromeOrange clone J.33, anti-CD34-PC7 clone 581 (both from Beckman Coulter), anti-CD56-APC-Vio770 clone REA196 (Miltenyi Biotec) and 7-AAD dye (Sigma Aldrich, Zwijndrecht, NL).

Flow cytometry-based effector cell potency assay

Effector cells potency is analysed by co-culturing effector cells and target tumour cell lines overnight and subsequently determining target cell killing via 7-AAD staining and flow cytometry. Target cells are resuspended at 1*10⁷ cells/ml in PBS and pre-labelled with 0.012 mg/ml Pacific Blue™ succinimidyl ester (PBSE, Thermo Fisher Scientific) for 10 minutes in a cell incubator; labelling is stopped by adding 1 volume of target cells culture medium supplemented with 10% FBS. Cells are then washed twice with PBS, counted via flow cytometry and finally resuspended at 1*10⁶ cells/ml in culture medium with 10% FBS. Effector cells are stained with cell-specific surface markers and counted via flow cytometry, then finally resuspended at 1*10⁶ cells/ml in GBGM supplemented with 2% HS. Pre-stained target cells are then co-cultured with effector cells in 96-well tissue culture plates at 1:1 ratio, 5*10⁴ cells/well for each cell type, in a volume of 100 μl, in technical triplicates. Effector cells alone and target cells alone are included as controls. After overnight co-culture in a cell incubator (37° C., 95% humidity, 5% CO₂), samples are diluted 1:1 with FACS buffer, stained with 7-AAD and immediately analysed via flow cytometry. Target cells are distinguished from effector cells through FSC/SSC properties and PBSE positivity; PBSE positive, 7-AAD negative or positive cells (PBSE/7-AAD⁻, i.e. viable target cells or PBSE17-AAD+, i.e. dead target cells) are then counted. The average of PBSE/7-AAD⁻ cells from the co-culture triplicates is normalised on the same value obtained for target cells cultured alone to exclude the influence of physiological death of target cells, then the percentage of killed cells is calculated by subtracting the obtained value to 1 and multiplying it by 100.

Statistical Analysis

All statistical analysis is performed using the GraphPad Prism 8 software (Sand Diego, Calif.). Data is represented as average of independent biological donors (N=2), error is calculated as standard deviation (SD). Student's t-test or one-way ANOVA with multiple comparisons correction (Tukey's method) are performed and significance of results is showed as p-values (ns, statistically non-significant: p>0.12; * p≤0.033; ** p≤0.002; *** p≤0.001).

Results

Part 1—Effect of CD34⁺ haematopoietic stem cell (HSC) progenitors' seeding density on in vitro cell expansion in 2% serum, 1x cytokines and LDC-free culture medium

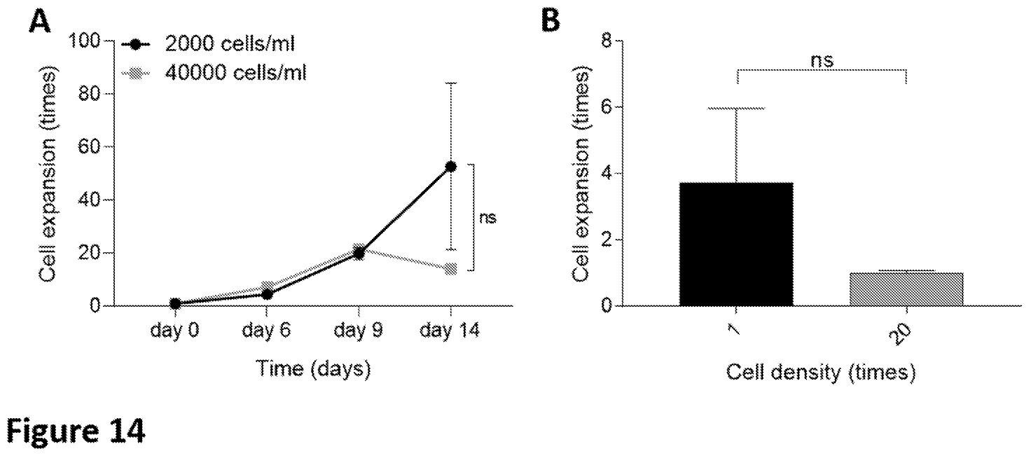

Day 0 human CD34⁺ HSCs isolated from fresh UCB, counted via flow cytometry and diluted at 2 densities, low (2000 CD34⁺ cells/ml) or high (40000 CD34⁺ cells/ml), are seeded for expansion culture in 2% serum, 1x cytokines and LDC-free medium (Table 4). Cultures are monitored and medium is refreshed regularly until day 14; intermediate and final cell expansion is analysed via flow cytometry at days 6, 9 and 14. For every time point, expansion is calculated on day 0 initial cell density and the average expansion of the two conditions is plotted against time (FIG. 14A). Observed cell expansion is analogous to what observed in LDC-free medium (as shown in Example 3) and in 2% serum, 5x cytokines, LDC-free medium (as shown in Example 4). Similarly, it is increased by low density seeding. Endpoint expansion reaches on average 53 times for 2000 CD34⁺ cells/ml or 14 times for 40000 CD34⁺ cells/ml. Inverse correlation between cell density and relative expansion is showed in FIG. 14B. These data demonstrate how, even if serum reduction in the medium is not compensated by increasing cytokines, low density seeding still gives an advantage in cell expansion.

Part 2—Effect of CD34⁺ haematopoietic stem cell (HSC) progenitors' expansion density on effector cells differentiation and potency in 2% serum, 1x cytokines and LDC-free culture medium

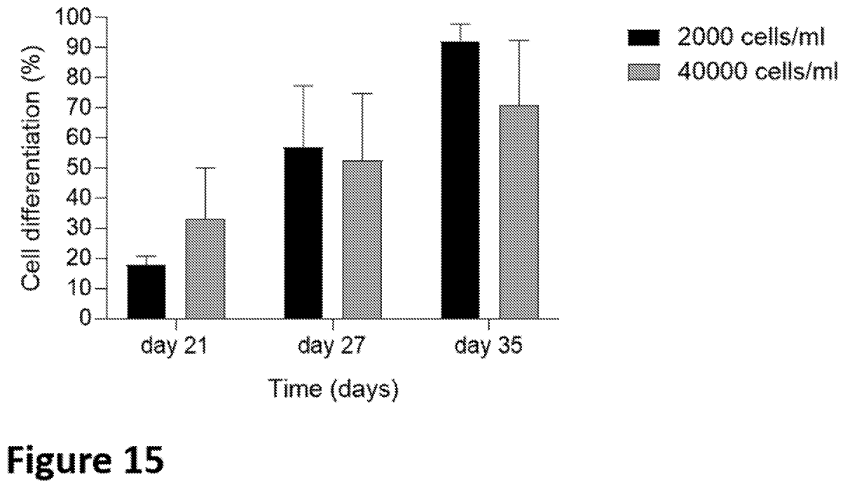

For differentiation phase, day 14 cells are set at the density of 1.5*10⁶ cells/ml in 2% serum, 1x cytokines and LDC-free differentiation medium. The ability of the cells to differentiate into mature effector cells and to further expand is monitored every week until day 35 and medium is refreshed twice every week. FIG. 15 shows the progression in cell differentiation from day 21 to 35 (from 15-45% at day 21 to 55-96% at day 35). Although initial being slower in differentiation, an advantage is observed at later stages for cells expanded as 2000 CD34⁺ cells/ml when compared to 40,000 CD34⁺ cells/ml. Day 35 expansion, similarly, is higher at lower density (not shown).

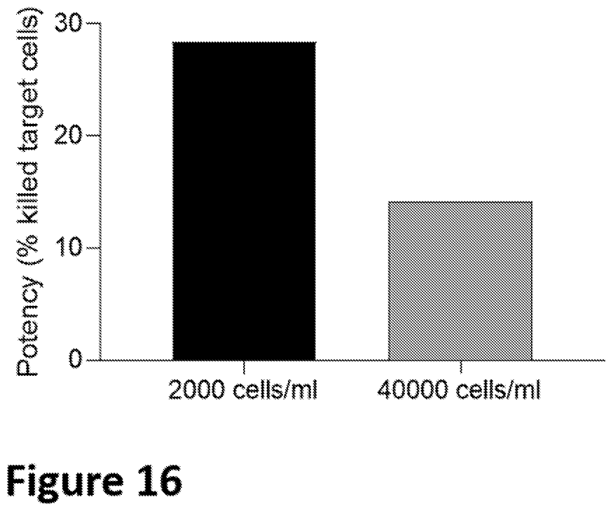

Effector cells potency, tested at day 35, shows how low-density seeding gives an advantage in the killing of target tumour cells (FIG. 16). For one tested donor, 28% of target cells are killed by 2000 CD34⁺ cells/ml effector cells after overnight co-culture in a 1:1 E:T ratio, where 14% are killed by 40000 CD34⁺ cells/ml effector cells (2-fold increase in cytotoxic potency). This example demonstrates how a strong impairment of culture conditions like serum withdrawal in combination with low cytokines affects cell expansion, differentiation and phenotype to a different extent. Nevertheless, initial culturing at low-density gives these cells a consistent advantage over high-density cultivated CD34⁺ stem cells.

TABLE 5
Day 0 seeding conditions of CD34⁺ haematopoietic stem cells (HSCs)
purified from fresh umbilical cord blood (UCB) and cultured in 2%
serum, 1× cytokines and LDC-free medium. Freshly isolated HSCs are
counted via flow cytometry and diluted at 2 different, increasing cell
concentrations (expressed in cells/ml) in 6-wells tissue culture plates
(as single replicates) in a volume of 2 ml/well. The column
“Concentration” shows the relative cell density between all
conditions, where the lowest, 2000 CD34⁺ cells/ml, is set as 1.

| | | day 0 |
|---|---|---|
| CD34⁺ HSC density | Concentration | volume |
| (cells/ml) | (times) | (ml) |
| 2000 CD34⁺ cells/ml | 1 | 2 |
| 40000 CD34⁺ cells/ml | 20 | 2 |

### REFERENCES

1. A. Gratwohl et al., Hematopoietic stem cell transplantation: a global perspective. Jama 303, 1617-1624 (2010).
2. J. Jansen, The first successful allogeneic bone-marrow transplant: Georges Mathe. Transfusion medicine reviews 19, 246-248 (2005).
3. M. Korbling, P. Anderlini, Peripheral blood stem cell versus bone marrow allotransplantation: does the source of hematopoietic stem cells matter? Blood 98, 2900-2908 (2001).
4. E. Dzierzak, N. A. Speck, Of lineage and legacy: the development of mammalian hematopoietic stem cells. Nature immunology 9, 129-136 (2008).
5. S. J. Morrison, A. C. Spradling, Stem cells and niches: mechanisms that promote stem cell maintenance throughout life. Cell 132, 598-611 (2008).
6. A. Wilson, A. Trumpp, Bone-marrow haematopoietic-stem-cell niches. Nat Rev Immunol 6, 93-106 (2006).
7. H. Kawamoto, T. Ikawa, K. Masuda, H. Wada, Y. Katsura, A map for lineage restriction of progenitors during hematopoiesis: the essence of the myeloid-based model. Immunol Rev 238, 23-36 (2010).
8. E. A. Copelan, Hematopoietic stem-cell transplantation. N Engl j Med 354, 1813-1826 (2006).
9. R. R. Jenq, M. R. van den Brink, Allogeneic haematopoietic stem cell transplantation: individualized stem cell and immune therapy of cancer. Nat Rev Cancer 10, 213-221 (2010).
10. L. Ruggeri et al., Effectiveness of donor natural killer cell alloreactivity in mismatched hematopoietic transplants. Science 295, 2097-2100 (2002).
11. J. A. Olson et al., NK cells mediate reduction of GVHD by inhibiting activated, alloreactive T cells while retaining GVT effects. Blood 115, 4293-4301 (2010).
12. W. D. Shlomchik et al., Prevention of graft versus host disease by inactivation of host antigen-presenting cells. Science 285, 412-415 (1999).
13. M. Tanaka et al., The impact of the dose of natural killer cells in the graft on severe acute graft-versus-host disease after unrelated bone marrow transplantation. Leukemia research 36, 699-703 (2012).
14. R. Kiessling, E. Klein, H. Pross, H. Wigzell, “Natural” killer cells in the mouse. II. Cytotoxic cells with specificity for mouse Moloney leukemia cells. Characteristics of the killer cell. Eur J Immunol 5, 117-121 (1975).
15. R. B. Herberman, M. E. Nunn, D. H. Lavrin, Natural cytotoxic reactivity of mouse lymphoid cells against syngeneic acid allogeneic tumors. I. Distribution of reactivity and specificity. Int J Cancer 16, 216-229 (1975).
16. R. B. Herberman, M. E. Nunn, H. T. Holden, D. H. Lavrin, Natural cytotoxic reactivity of mouse lymphoid cells against syngeneic and allogeneic tumors. II. Characterization of effector cells. Int J Cancer 16, 230-239 (1975).
17. R. Kiessling, E. Klein, H. Wigzell, “Natural” killer cells in the mouse. I. Cytotoxic cells with specificity for mouse Moloney leukemia cells. Specificity and distribution according to genotype. Eur J Immunol 5, 112-117 (1975).
18. E. Vivier, E. Tomasello, M. Baratin, T. Walzer, S. Ugolini, Functions of natural killer cells. Nat Immunol 9, 503-510 (2008).
19. M. A. Caligiuri, Human natural killer cells. Blood 112, 461-469 (2008).
20. M. A. Cooper, T. A. Fehniger, A. Fuchs, M. Colonna, M. A. Caligiuri, NK cell and DC interactions. Trends in immunology 25, 47-52 (2004).
21. G. Ferlazzo, C. Munz, NK cell compartments and their activation by dendritic cells. J Immunol 172, 1333-1339 (2004).
22. M. J. Robertson, J. Ritz, Biology and clinical relevance of human natural killer cells. Blood 76, 2421-2438 (1990).
23. L. L. Lanier, R. Testi, J. Bind!, J. H. Phillips, Identity of Leu-19 (CD56) leukocyte differentiation antigen and neural cell adhesion molecule. J Exp Med 169, 2233-2238 (1989).
24. L. L. Lanier, A. M. Le, C. I. Civin, M. R. Loken, J. H. Phillips, The relationship of CD16 (Leu-11) and Leu-19 (NKH-1) antigen expression on human peripheral blood NK cells and cytotoxic T lymphocytes. J Immunol 136, 4480-4486 (1986).
25. L. L. Lanier, NK cell recognition. Annu Rev Immunol 23, 225-274 (2005).
26. L. L. Lanier, J. H. Phillips, J. Hackett, Jr., M. Tutt, V. Kumar, Natural killer cells: definition of a cell type rather than a function. J Immunol 137, 2735-2739 (1986).
27. J. A. Trapani, M. J. Smyth, Functional significance of the perforin/granzyme cell death pathway. Nat Rev Immunol 2, 735-747 (2002).
28. G. Bossi, G. M. Griffiths, CTL secretory lysosomes: biogenesis and secretion of a harmful organelle. Semin Immunol 17, 87-94 (2005).
29. V. Screpanti, R. P. Wallin, A. Grandien, H. G. Ljunggren, Impact of FASL-induced apoptosis in the elimination of tumor cells by NK cells. Molecular immunology 42, 495-499 (2005).
30. L. Moretta, A. Moretta, Unravelling natural killer cell function: triggering and inhibitory human NK receptors. EMBO J 23, 255-259 (2004).
31. D. H. Raulet, Interplay of natural killer cells and their receptors with the adaptive immune response. Nature immunology 5, 996-1002 (2004).
32. E. Vivier, J. A. Nunes, F. Vely, Natural killer cell signaling pathways. Science 306, 1517-1519 (2004).
33. L. L. Lanier, NK cell receptors. Annu Rev Immunol 16, 359-393 (1998).
34. A. Moretta et al., Activating receptors and coreceptors involved in human natural killer cell-mediated cytolysis. Annu Rev Immunol 19, 197-223 (2001).
35. R. Willemze et al., KIR-ligand incompatibility in the graft-versus-host direction improves outcomes after umbilical cord blood transplantation for acute leukemia. Leukemia 23, 492-500 (2009).
36. N. D. Huntington, C. A. Vosshenrich, J. P. Di Santo, Developmental pathways that generate natural-killer-cell diversity in mice and humans. Nat Rev Immunol 7, 703-714 (2007).
37. A. G. Freud et al., A human CD34(+) subset resides in lymph nodes and differentiates into CD56bright natural killer cells. Immunity 22, 295-304 (2005).
38. A. G. Freud et al., Evidence for discrete stages of human natural killer cell differentiation in vivo. J Exp Med 203, 1033-1043 (2006).
39. T. A. Fehniger et al., CD56bright natural killer cells are present in human lymph nodes and are activated by T cell-derived IL-2: a potential new link between adaptive and innate immunity. Blood 101, 3052-3057 (2003).
40. G. Ferlazzo et al., The abundant NK cells in human secondary lymphoid tissues require activation to express killer cell Ig-like receptors and become cytolytic. J Immunol 172, 1455-1462 (2004).
41. D. N. Eissens et al., CD3+/CD19+-depleted grafts in HLA-matched allogeneic peripheral blood stem cell transplantation lead to early NK cell cytolytic responses and reduced inhibitory activity of NKG2A. Leukemia 24, 583-591 (2010).
42. H. G. Shilling et al., Reconstitution of NK cell receptor repertoire following HLA-matched hematopoietic cell transplantation. Blood 101, 3730-3740 (2003).
43. J. P. Di Santo, Natural killer cell developmental pathways: a question of balance. Annu Rev Immunol 24, 257-286 (2006).
44. S. Kim et al., Licensing of natural killer cells by host major histocompatibility complex class I molecules. Nature 436, 709-713 (2005).
45. D. H. Raulet, R. E. Vance, Self-tolerance of natural killer cells. Nat Rev Immunol 6, 520-531 (2006).
46. N. Anfossi et al., Human NK cell education by inhibitory receptors for MHC class I. Immunity 25, 331-342 (2006).
47. N. C. Fernandez et al., A subset of natural killer cells achieves self-tolerance without expressing inhibitory receptors specific for self-MHC molecules. Blood 105, 4416-4423 (2005).
48. S. Cooley et al., A subpopulation of human peripheral blood NK cells that lacks inhibitory receptors for self-MHC is developmentally immature. Blood 110, 578-586 (2007).
49. T. A. Fehniger et al., Differential cytokine and chemokine gene expression by human NK cells following activation with IL-18 or IL-15 in combination with IL-12: implications for the innate immune response. J Immunol 162, 4511-4520 (1999).
50. M. A. Cooper, T. A. Fehniger, M. A. Caligiuri, The biology of human natural killer-cell subsets. Trends Immunol 22, 633-640 (2001).
51. A. G. Freud, M. A. Caligiuri, Human natural killer cell development. Immunol Rev 214, 56-72 (2006).
52. S. S. Farag, M. A. Caligiuri, Human natural killer cell development and biology. Blood Rev 20, 123-137 (2006).
53. I. Douagi, F. Colucci, J. P. Di Santo, A. Cumano, Identification of the earliest prethymic bipotent T/NK progenitor in murine fetal liver. Blood 99, 463-471 (2002).
54. H. Yu et al., Flt3 ligand promotes the generation of a distinct CD34(+) human natural killer cell progenitor that responds to interleukin-15. Blood 92, 3647-3657 (1998).
55. G. Carayol et al., NK cells differentiated from bone marrow, cord blood and peripheral blood stem cells exhibit similar phenotype and functions. Eur J Immunol 28, 1991-2002 (1998).
56. M. 0. Muench et al., Differential effects of interleukin-3, interleukin-7, interleukin 15, and granulocyte-macrophage colony-stimulating factor in the generation of natural killer and B cells from primitive human fetal liver progenitors. Exp Hematol 28, 961-973 (2000).
57. J. S. Miller, K. A. Alley, P. McGlave, Differentiation of natural killer (NK) cells from human primitive marrow progenitors in a stroma-based long-term culture system: identification of a CD34+7+ NK progenitor. Blood 83, 2594-2601 (1994).
58. E. E. Rosmaraki et al., Identification of committed NK cell progenitors in adult murine bone marrow. Eur J Immunol 31, 1900-1909 (2001).
59. I. M. Bennett et al., Definition of a natural killer NKR-P1A+/CD56-/CD16- functionally immature human NK cell subset that differentiates in vitro in the presence of interleukin 12. J Exp Med 184, 1845-1856 (1996).
60. S. A. Perez et al., A novel myeloid-like NK cell progenitor in human umbilical cord blood. Blood 101, 3444-3450 (2003).
61. B. Grzywacz, N. Kataria, B. R. Blazar, J. S. Miller, M. R. Verneris, Natural killer-cell differentiation by myeloid progenitors. Blood 117, 3548-3558 (2011).
62. K. Barton et al., The Ets-1 transcription factor is required for the development of natural killer cells in mice. Immunity 9, 555-563 (1998).
63. F. Colucci et al., Differential requirement for the transcription factor PU.1 in the generation of natural killer cells versus B and T cells. Blood 97, 2625-2632 (2001).
64. H. D. Lacorazza et al., The ETS protein MEF plays a critical role in perforin gene expression and the development of natural killer and NK-T cells. Immunity 17, 437-449 (2002).
65. S. I. Samson et al., GATA-3 promotes maturation, IFN-gamma production, and liver-specific homing of NK cells. Immunity 19, 701-711 (2003).
66. M. J. Townsend et al., T-bet regulates the terminal maturation and homeostasis of NK and Valpha14i NKT cells. Immunity 20, 477-494 (2004).
67. S. Taki, S. Nakajima, E. Ichikawa, T. Saito, S. Hida, IFN regulatory factor-2 deficiency revealed a novel checkpoint critical for the generation of peripheral NK cells. J Immunol 174, 6005-6012 (2005).
68. M. D. Boos, Y. Yokota, G. Eberl, B. L. Kee, Mature natural killer cell and lymphoid tissue-inducing cell development requires Id2-mediated suppression of E protein activity. J Exp Med 204, 1119-1130 (2007).
69. T. Ikawa, S. Fujimoto, H. Kawamoto, Y. Katsura, Y. Yokota, Commitment to natural killer cells requires the helix-loop-helix inhibitor Id2. Proc Natl Acad Sci U S A 98, 5164-5169 (2001).
70. D. M. Gascoyne et al., The basic leucine zipper transcription factor E4BP4 is essential for natural killer cell development. Nat Immunol 10, 1118-1124 (2009).
71. N. E. Lemos et al., Quantification of peripheral blood CD34(+) cells prior to stem cell harvesting by leukapheresis: a single center experience. Hematol Transfus Cell Ther 40, 213-218 (2018).
72. T. F. Wang et al., Factors associated with peripheral blood stem cell yield in volunteer donors mobilized with granulocyte colony-stimulating factors: the impact of donor characteristics and procedural settings. Biology of blood and marrow transplantation : journal of the American Society for Blood and Marrow Transplantation 14, 1305-1311 (2008).
73. L. E. Shields, R. G. Andrews, Gestational age changes in circulating CD34⁺ hematopoietic stem/progenitor cells in fetal cord blood. Am J Obstet Gynecol 178, 931-937 (1998).
74. K. Vettenranta, I. Piirto, U. M. Saarinen-Pihkala, The effects of the mode of delivery on the lymphocyte composition of a placental/cord blood graft. Journal of hematotherapy 6, 491-493 (1997).
75. D. Grisaru et al., Placing the newborn on the maternal abdomen after delivery increases the volume and CD34 cell content in the umbilical cord blood collected: an old maneuver with new applications. Am J Obstet Gynecol 180, 1240-1243 (1999).
76. S. R. Panch, J. Szymanski, B. N. Savani, D. F. Stroncek, Sources of Hematopoietic Stem and Progenitor Cells and Methods to Optimize Yields for Clinical Cell Therapy. Biology of blood and marrow transplantation : journal of the American Society for Blood and Marrow Transplantation 23, 1241-1249 (2017).
77. J. E. Wagner, Jr. et al., Phase I/II Trial of StemRegenin-1 Expanded Umbilical Cord Blood Hematopoietic Stem Cells Supports Testing as a Stand-Alone Graft. Cell stem cell 18, 144-155 (2016).
78. M. E. Horwitz et al., Umbilical cord blood expansion with nicotinamide provides long-term multilineage engraftment. J Clin Invest 124, 3121-3128 (2014).
79. M. de Lima et al., Cord-blood engraftment with ex vivo mesenchymal-cell coculture. N Engl J Med 367, 2305-2315 (2012).

1. A method for producing a collection of stem cells, progenitor cells, and/or NK cells, said method comprising the step of
(i) initiating a cell culture from a sample comprising CD34^{÷} human stem cells and culturing the cells for at least 7 days in a basic culture medium comprising stem cell factor (SCF) and interleukin-7 (IL-7), and one or more of flt-3Ligand (FLT-3L) and thrombopoietin (TPO), characterized in that the cell culture is initiated at a cell density of 12,000 CD34′ cells/ml or less.

2. The method according to claim 1, wherein the cell culture is initiated at a cell density of between 500 and 10,000 CD34′ cells/ml more preferably between 1,000 and 8,000 CD34⁺ cells/ml, more preferably between 2,000 and 6,000 CD34±cells/ml.

3. The method according to claim 1 wherein said method further comprises the step of
(ii) culturing cells obtained in step (i) for at least 4 days in a medium comprising IL-15 and IL-7, and one or more of SCF or FLT-3L.

4. The method according, to claim 3, wherein said method further comprises the step of
(iii) culturing cells obtained in step (ii) for at least 13 days in a culture medium comprising a collection of cytokines, wherein said collection of cytokines comprises three or more of SCF, IL-7. IL-15 and IL-2, thereby obtaining a collection of cultured cells containing a plurality of NK cells.

5. The method according to claim 1, wherein the method results in an at least 150-fold expansion of cells, preferably at least 200-fold, more preferably at least 300-fold, most preferably at least 500-fold at day 12.

6. The method according to claim 1, wherein the method results in an at least 150-fold expansion of cells, preferably at least 200-fold, more preferably at least 400-fold, most preferably at least 800-fold at day 15.

7. The method according to claim 1, wherein the sample comprising CD34⁺ stem cells is obtained from human cord blood.

8. The method according to claim 1, wherein the sample comprising CD34⁺ stein cells is obtained by selecting CD34⁺ human stem cells (HSC) through a fully automated closed system.

9. The method according to claim 1, wherein the sample comprising CD34⁺ stem cells is obtained by selecting CD34⁺ human stem cells (HSC) through manual column separation.

10. The method according to claim 1, wherein, independently from one another, and if present, SCF is present at concentration between 2 ng/ml and 200 ng/ml, Flt3-L at concentration between 2 ng/ml and 200 ng/ml, TPO at concentration between 2 ng/ml and 200 ng/ml, IL-7 at concentration between 2 ng/ml and 200 ng/ml, IL-15 at a concentration between 2 ng/ml and 200 ng/ml, M-2 at a concentration of between 100-10,000

11. The method according to claim 1, wherein the NK cells obtained comprise at least 50%, preferably at least 60%, more preferably at least 75%, most preferably at least 80% fully differentiated NK cells after 28 days of culture.

12. The method according to claim 1, wherein the NK cells obtained comprise at least 75%, more preferably at least 80%, more preferably at least 85%, most preferably at least 90% fully differentiated NK cells after 35 days of culture.

13. The method according to claim 1, wherein the NK cells obtained are able to kill at least 30%, preferably at least 40%, most preferably at least 45% of their target cells, when measured in a cell cytotoxicity assay using K562 cells in a 1 effector cell to 1 target cell ratio.

14. A collection of NK cells obtained from method according to claim 1.

15. A collection of NK cells according to claim 14, wherein the collection comprises at least 10.000.000.000 cells from a single donor.

16. A Pharmaceutical composition, comprising a collection of NK cells according to claim 1.

17. (canceled)

18. (canceled)

19. The method for treating an individual in need of immunotherapy, the method comprising administering to the individual a pharmaceutical composition according to claim 16. wherein the composition comprises a therapeutically effective amount of CD56±CD3-cells.

20. The method for treating an individual accord o claim 17, wherein the treatment is for the treatment of a tumour.

21. The method for treating an individual according to claim 18, wherein the tumour is a hematopoietic or lymphoid tumour or wherein the tumour is a solid tumour.
